(19) United States
(12) Patent Application Publication
(10) Pub. No.: US 2026/0209221 A1
(43) Pub. Date: Jul. 23, 2026

(54) SOLID FORMS OF {5-CYCLOPROPYL-2-[2-(3,6-DIFLUORO¬PYRIDIN-2-YLAMINO)-PYRIDIN-4-YL]-PYRIDO[3,4-D]PYRIMIDIN-4-YL }-( (S)-3,3-DIMETHYL-PIPERIDIN-4-YL)-AMINE

(71) Applicant: WINDTREE THERAPEUTICS, INC., Warrington, PA (US)

(72) Inventor: John C. AMEDIO, Franklin, MA (US)

(73) Assignee: WINDTREE THERAPEUTICS, INC., Warrington, PA (US)

(21) Appl. No.: 19/139,166

(22) PCT Filed: Dec. 12, 2023
(86) PCT No.: PCT/US2023/083637

§ 371 Date: Jun. 13, 2025

Related U.S. Application Data
(60) Provisional application No. 63/387,200, filed on Dec. 13, 2022.

(51) Int. Cl. C07D 471/04

(52) U.S. Cl. CPC C07D 471/04

(57) ABSTRACT

Disclosed herein are solid forms (e.g., crystalline forms) of 4-(5-cyclopropyl-4-piperazin-1-yl-pyrido[3,4-d]pyrimidin-2-yl)-9H-pyrido[2,3-b]indole (Compound 1), and compositions, pharmaceutical compositions, kits, methods, and uses thereof. The compounds disclosed herein are inhibitors of atypical protein kinase C (aPKC) and their solid forms are therefore useful for the treatment and/or prevention of basal cell carcinoma.

### CROSS REFERENCE TO RELATED APPLICATIONS

This application claims priority under 35 U.S.C. § 119(e) to U.S. Provisional Application Ser. No. 63/387,200, filed on Dec. 13, 2022, the contents of which are incorporated herein by reference in their entirety.

### BACKGROUND

Polymorphic forms of a particular compound occur where the same compound crystallizes in different lattice arrangements. The resulting polymorphs may have distinct thermodynamic properties and stabilities. In addition, the crystalline polymorph form can impact solubility, storage stability, ease of preparation and formulation, and in vivo pharmacology. Therefore, in cases where two or more polymorphic forms can be produced, it is desirable to obtain each in substantially pure form. There is an ongoing need for new or purer polymorphic forms of therapeutically active compounds to obtain improved physical properties and bioavailability.

### SUMMARY OF THE INVENTION

Disclosed herein are crystalline forms of Compound 1,

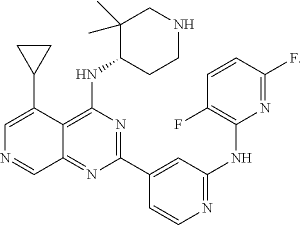 [SMILES: CC1(C)CNCC[C@@H]1Nc1nc(-c2ccnc(Nc3nc(F)ccc3F)c2)nc2cncc(C3CC3)c12]

In one aspect, the present disclosure provides a crystalline Form II of Compound 1:

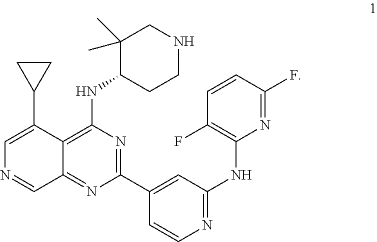 [SMILES: CC1(C)CNCC[C@@H]1Nc1nc(-c2ccnc(Nc3nc(F)ccc3F)c2)nc2cncc(C3CC3)c12]

In another aspect, the present disclosure provides a composition comprising the crystalline Form II of Compound 1, wherein the composition comprises greater than or equal to 90% by weight of the crystalline Form II of Compound 1.

In another aspect, the present disclosure provides a composition comprising the crystalline Form II of Compound 1, wherein the molar ratio of the amount of Form II of Compound 1 to the sum of amounts of other forms in the composition is equal to or greater than 90:10.

In another aspect, the present disclosure provides a crystalline Form III of Compound 1:

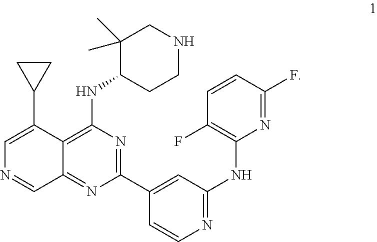 [SMILES: CC1(C)CNCC[C@@H]1Nc1nc(-c2ccnc(Nc3nc(F)ccc3F)c2)nc2cncc(C3CC3)c12]

In another aspect, the present disclosure provides a composition comprising the crystalline Form III of Compound 1, wherein the composition comprises greater than or equal to 90% by weight of the crystalline Form III of Compound 1.

In another aspect, the present disclosure provides a composition comprising the crystalline Form III of Compound 1, wherein the molar ratio of the amount of Form III of Compound 1 to the sum of amounts of other forms in the composition is equal to or greater than 90:10.

In another aspect, the present disclosure provides a pharmaceutical composition comprising a crystalline form provided herein, or a composition provide herein, and a pharmaceutically acceptable excipient.

In another aspect, the present disclosure provides a method of preparing a crystalline Form II of Compound 1, or a composition thereof, the method comprising the steps of:
(1) providing a solution of Compound 1 in one or more solvents;
(2) precipitating the crystalline Form II of Compound 1 from the solution of Compound 1; and
(3) isolating the crystalline Form II of Compound 1.

In another aspect, the present disclosure provides a method of preparing a crystalline Form III of Compound 1, or a composition thereof, the method comprising the steps of:
(1) providing a solution of Compound 1 in one or more solvents;
(2) precipitating the crystalline Form III of Compound 1 from the solution of Compound 1; and
(3) isolating the crystalline Form III of Compound 1.

In another aspect, the present disclosure provides a method for treating cancer in a subject in need thereof, comprising administering to the subject a crystalline form provided herein, a composition thereof, or a pharmaceutical composition thereof.

In another aspect, the present disclosure provides a crystalline form provided herein, a composition thereof, or a pharmaceutical composition thereof for use in treating cancer in a subject in need thereof.

In another aspect, the present disclosure provides a use of a crystalline form provided herein, a composition thereof, or a pharmaceutical composition thereof for the treatment of cancer in a subject in need thereof.

In another aspect, the present disclosure provides a use of a crystalline form provided herein, a composition thereof, or a pharmaceutical composition thereof in the manufacture of a medicament for the treatment of basal cell carcinoma, solid tumors, or acute myeloid leukemia in a subject in need thereof.

In another aspect, the present disclosure provides a kit comprising a crystalline form provided herein, a composition thereof, or a pharmaceutical composition thereof, and instructions for using the crystalline form, composition, or pharmaceutical composition.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects disclosed herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

FIG. 1 shows an X-ray powder diffraction (XPRD) pattern for Form II of Compound 1.

FIG. 2 shows a differential scanning calorimetry (DSC) thermogram of Form II of Compound 1.

FIG. 3 shows a thermogravimetric analysis (TGA) thermogram of Form II of Compound 1.

FIG. 4 shows an XPRD pattern for Form III of Compound 1.

FIG. 5 shows a DSC thermogram of Form III of Compound 1.

FIG. 6 shows a TGA thermogram of Form III of Compound 1.

### DEFINITIONS

### Compound 1:

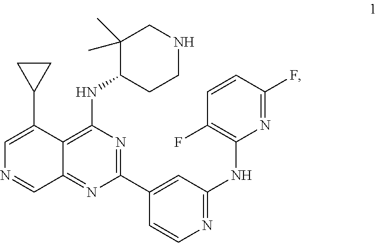 [SMILES: CC1(C)CNCC[C@@H]1Nc1nc(-c2ccnc(Nc3nc(F)ccc3F)c2)nc2cncc(C3CC3)c12]

is also referred to herein as “CRT0329868” and “4-(5-cyclopropyl-4-piperazin-1-yl-pyrido[3,4-d]pyrimidin-2-yl)-9H-pyrido[2,3-b]indole.”

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. Describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. In some embodiments, this physical association includes hydrogen bonding. Conventional solvents include water, methanol, ethanol, acetic acid, DMSO, THF, diethyl ether, and the like. In some embodiments, the compounds described herein are be prepared, e.g., in crystalline form, and are solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, in some embodiments, a hydrate of a compound is represented, for example, by the general formula R·x H₂O, wherein R is the compound, and x is a number greater than 0. In some embodiments, a given compound forms more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. In some embodiments, an enantiomer is characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). In some embodiments, a chiral compound exists as either individual enantiomer or as a mixture thereof. A mixture containing equal proportions of the enantiomers is called a “racemic mixture”.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. In some embodiments, tautomerizations (i.e., the reaction providing a tautomeric pair) are catalyzed by acid or base. Exemplary tautomerizations include keto-to-enol, amide-to-imide, lactam-to-lactim, enamine-to-imine, and enamine-to-(a different enamine) tautomerizations.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 2θ of, e.g., between 2° and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 2θ of between 2° and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point). The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. In some embodiments, co-crystals are useful to improve the properties (e.g., solubility, stability, and ease of formulation) of a compound disclosed herein.

The term “polymorph” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. In some embodiments, recrystallization solvent, rate of crystallization, storage temperature, and other factors cause one crystal form to dominate. In some embodiments, various polymorphs of a compound are prepared by crystallization under different conditions.

When a characteristic peak of an X-ray powder diffraction pattern is expressed in “degrees 2-theta (±0.2)” at Z, where Z is a number, the characteristic peak is at between Z+0.2 and Z−0.2 degrees 2-theta, inclusive.

The term “impurity” refers to extraneous matter included in a desired substance (e.g., a compound (e.g., Compound 1), or a salt, solvate, hydrate, co-crystal, amorphous form, or crystalline form thereof). Extraneous matter includes one or more substances that are different from the desired substance. In certain embodiments, the extraneous matter is undesired extraneous matter. For example, when the desired substance is a crystalline compound, an amorphous form of the compound included in or with the crystalline compound is an impurity. For example, when the desired substance is a crystalline compound, a different crystalline form of the compound included in or with the crystalline compound is an impurity.

The term “substantially Y,” where Y is a characteristic (e.g., anhydrous), refers to a characteristic that is at least 99.5%, at least 99%, at least 98%, at least 97%, at least 96%, at least 95%, at least 92%, or at least 90% the same as Y, unless expressly provided otherwise.

The term “substantially free of impurities” means that a desired substance does not contain a significant amount of extraneous matter (e.g., undesired extraneous matter). What amount of the extraneous matter constitutes a significant amount depends on the subject matter and is understood in the art. In certain embodiments, at least 0.5%, at least 1%, at least 2%, at least 3%, at least 4%, at least 5%, at least 10%, at least 20%, or at least 30% by weight of extraneous matter in the desired substance is a significant amount of extraneous matter. In some embodiments, the amount of impurities is determined using high-performance liquid chromatography (HPLC) with, e.g., an ultraviolet (UV) detector at, e.g., about 214 or about 220 nm. In some embodiments, under suitable conditions, a desired substance and each impurity are separated after HPLC, and the areas of the peaks of the resulting HPLC chromatogram are determined. In certain embodiments, the weight ratio of the amount of an impurity to the amount of a desired substance is the ratio of the peak area of the impurity to the peak area of the desired substance.

In some embodiments, when a polymorphic form is described, “substantially free” is meant to refer to the identified polymorph as described herein, which is substantially free of any other polymorph. “Substantially free of” another polymorph indicates at least a 70/30 molar ratio of the two polymorphs, more preferably 80/20, 90/10, 95/5, 97/3, 98/2, 99/1, or more. In some embodiments, one of the polymorphs will be present in an amount of at least 99/1.

The term “polar solvent” refers to a solvent with a large dipole moment or high dielectric constant. In some embodiments, the term “polar solvent” refers to an aprotic polar solvent, a polar protic solvent, or combinations thereof. In some embodiments, a polar solvent has either a permanent separation of positive and negative charges, or the centers of positive and negative charges do not coincide in the molecules. Polar solvents include, but are not limited to chloromethane, dichloromethane, dichloroethane, tetrahydrofuran, dimethylformamide, acetonitrile, nitromethane, propylene carbonate, formic acid, butanol, isopropanol, methyltetrahydrofuran, trifluoromethylbenzene, ethyl acetate, ethyl ether, acetone, dimethyl sulfoxide, alcohols, acetic acid, and esters.

The term “nonpolar solvent” refers to a solvent with a low dipole moment or small dielectric constant. In some embodiments, the solvent lacks significant partial charges on any atoms (e.g., hydrocarbons). In some embodiments, the solvent includes polar bonds are arranged in such a way that the effects of their partial charges are cancelled out (e.g., chloroform). Non-polar solvents include, but are not limited to, hydrocarbons, benzene, toluene, xylene, pentane, cyclohexane, n-hexanes, n-heptane, octane, iso-octane, chloroform, ether, dimethyl ether, diethyl ether, methyl-tert-butyl ether, 1,4-dioxane, and neutral or non-ionic surfactants.

The term “aprotic solvent” refers to a solvent that can neither accept nor donate a proton. Aprotic solvents include, but are not limited to, acetone, acetonitrile, benzene, butanone, butyronitrile, carbontetrachloride, chlorobenzene, chloroform, 1,2-dichloroethane, dichloromethane, diethyl ether, dimethylacetamide, N,N-dimethylformamide (DMF), dimethylsulfoxide (DMSO), 1,4-dioxane, ethyl acetate, ethylene glycol dimethyl ether, hexane, N-methyl-2-pyrrolidone, pyridine, tetrahydrofuran (THF), and toluene. Certain aprotic solvents are polar solvents. Examples of polar aprotic solvents include, but are not limited to, acetone, acetonitrile, butanone, N,N-dimethylformamide, and dimethylsulfoxide. Certain aprotic solvents are non-polar solvents. Examples of nonpolar, aprotic solvents include, but are not limited to, diethyl ether, aliphatic hydrocarbons, such as hexane, aromatic hydrocarbons, such as benzene and toluene, and symmetrical halogenated hydrocarbons, such as carbon tetrachloride.

The term “protic solvent” a solvent that contains a hydrogen atom bonded to an electronegative atom, such as an oxygen atom or a nitrogen atom. Protic solvents include, but are not limited to, carboxylic acids, such as acetic acid, alcohols (e.g., methanol, ethanol, isopropanol), amines, amides, and water.

The terms “pharmaceutical composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal.

The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. In some embodiments, a target tissue is an abnormal or unhealthy tissue. In some embodiments, a target tissue is an abnormal or unhealthy tissue, which needs to be treated. In some embodiments, a target tissue is a normal or healthy tissue that is under a higher than normal risk of becoming abnormal or unhealthy, which needs to be prevented. In certain embodiments, the target tissue is the skin. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound disclosed herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease disclosed herein. In some embodiments, treatment is administered after one or more signs or symptoms of the disease have developed or have been observed. In other embodiments, treatment is administered in the absence of signs or symptoms of the disease. For example, in some embodiments, treatment is administered to a susceptible subject prior to the onset of symptoms (e.g., in light of a history of symptoms and/or in light of exposure to a pathogen). In some embodiments, treatment is continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound disclosed herein refers to an amount sufficient to elicit the desired biological response. In some embodiments, an effective amount of a compound disclosed herein varies depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound disclosed herein in a single dose. In certain embodiments, an effective amount is the combined amounts of a compound disclosed herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as disclosed herein provide guidance for the administration of provided pharmaceutical compositions to an adult. In some embodiments, the amount to be administered to, for example, a child or an adolescent is determined by a medical practitioner or person skilled in the art. In some embodiments, the amount to be administered to, for example, a child or an adolescent is determined by a medical practitioner or person skilled in the art and is lower or the same as that administered to an adult.

A “therapeutically effective amount” of a compound disclosed herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. In some embodiments, the term “therapeutically effective amount” encompasses an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting atypical protein kinase C. In certain embodiments, a therapeutically effective amount is an amount sufficient for treating basal cell carcinoma. In certain embodiments, a therapeutically effective amount is an amount sufficient for inhibiting atypical protein kinase C and treating basal cell carcinoma.

A “prophylactically effective amount” of a compound disclosed herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. In some embodiments, the term “prophylactically effective amount” encompasses an amount that improves overall prophylaxis or enhances the prophylactic efficacy of another prophylactic agent. In certain embodiments, a prophylactically effective amount is an amount sufficient for inhibiting atypical protein kinase C. In certain embodiments, a prophylactically effective amount is an amount sufficient for preventing basal cell carcinoma. In certain embodiments, a prophylactically effective amount is an amount sufficient for inhibiting atypical protein kinase C and preventing basal cell carcinoma.

As used herein the term “inhibit” or “inhibition” in the context of kinases, for example, in the context of atypical protein kinase C, refers to a reduction in the activity of the kinase. In some embodiments, the term refers to a reduction of the level of activity, e.g., atypical protein kinase C activity, to a level that is statistically significantly lower than an initial level. In some embodiments, the term refers to a reduction of the level of activity, e.g., atypical protein kinase C activity, to a level that is statistically significantly lower than an initial level, which is a baseline level of activity. In some embodiments, the term refers to a reduction of the level of activity, e.g., atypical protein kinase C activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level. In some embodiments, the term refers to a reduction of the level of activity, e.g., atypical protein kinase C activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which is a baseline level of activity.

A “proliferative disease” refers to a disease that occurs due to abnormal growth or extension by the multiplication of cells (Walker, Cambridge Dictionary of Biology; Cambridge University Press: Cambridge, UK, 1990). In some embodiments, a proliferative disease is associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. In some embodiments, angiogenesis is chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. In some embodiments, a neoplasm or tumor is “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors later give rise to malignant neoplasms, such as those resulting from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. Myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values. In some embodiments, the term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X. For example, the term “about 100” refers to between 99.5 and 100.5, between 99 and 101, between 98 and 102, between 97 and 103, between 96 and 104, between 95 and 105, between 92 and 108, or between 90 and 110, inclusive.

Unless otherwise required by context, singular terms shall include pluralities, and plural terms shall include the singular.

### DETAILED DESCRIPTION OF CERTAIN EMBODIMENTS

Compound 1 has been found to exist in crystalline forms as described herein. Different crystalline forms of a compound (e.g., Compound 1) typically differ in their physical and/or chemical properties due to the arrangement of molecules in the solid form (e.g., the arrangement of molecules in a crystal lattice). In some embodiments, the different crystalline forms result in different pharmacokinetic and/or pharmacodynamic properties. In some embodiments, the different crystalline forms exhibit one or more of increased solubility, increased permeability, increased stability, increased ease of formulation, storage, transportation, and/or administration, decreased costs of formation, transportation, storage, and/or administration, increased adsorption, modified distribution, increased bioavailability, increased or decreased metabolism, increased or decreased excretion, increased potency, increased efficacy, decreased toxicity, decreased frequency and/or severity of side effects, and/or increased patient compliance relative to Compound 1, an amorphous form of Compound 1, an amorphous form of a salt of Compound 1, a different crystalline form of Compound 1, a different crystalline form of a salt of Compound 1, and/or a different co-crystal comprising Compound 1.

Different solid forms of a compound (e.g., Compound 1) are typically distinguished by X-ray diffraction, in particular X-ray powder diffraction (XRPD, obtained by, e.g., a method described herein), and by other methods, such as, differential scanning calorimetry (DSC, obtained by, e.g., a method described herein), thermal gravimetric analysis (TGA, obtained by, e.g., a method described herein), and/or solubility (e.g., thermodynamic solubility).

### Crystalline Form II

In one aspect, the present disclosure provides a crystalline Form II of Compound 1:

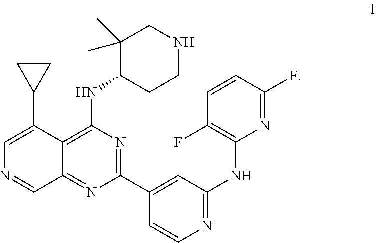 [SMILES: CC1(C)CNCC[C@@H]1Nc1nc(-c2ccnc(Nc3nc(F)ccc3F)c2)nc2cncc(C3CC3)c12]

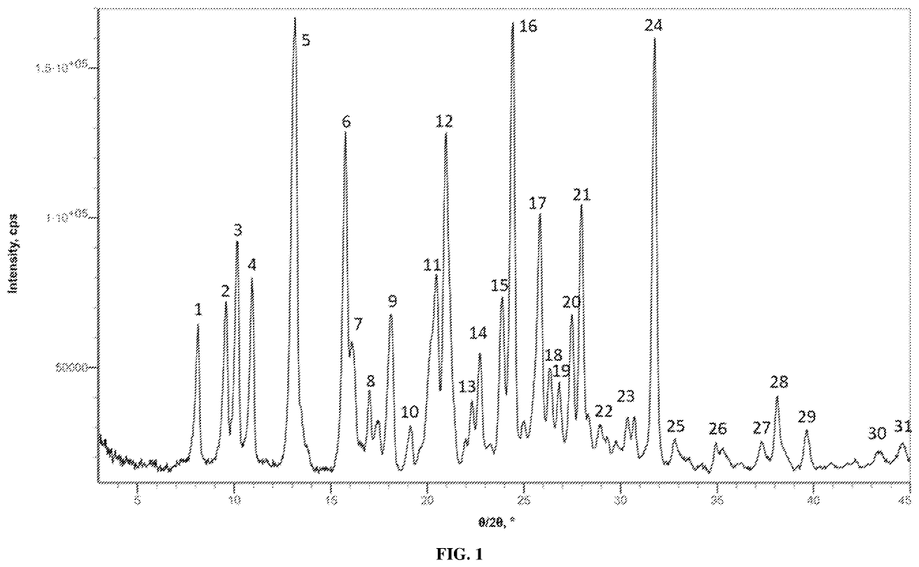

In some embodiments, the crystalline Form II of Compound 1 is characterized by an X-ray powder diffraction (XRPD) pattern substantially similar to that depicted in FIG. 1 when measured using CuK_{α} radiation. In some embodiments, the crystalline Form II of Compound 1 is characterized by an X-ray powder diffraction (XRPD) pattern substantially similar to that tabulated in Table 3.

In some embodiments, the crystalline Form II of Compound 1 is characterized by an XRPD pattern when measured using CuK_{α} radiation comprising characteristic peaks, expressed in degrees 2-theta (±0.2), at 8.1, 9.6, and 10.9. In some embodiments, the crystalline Form II of Compound 1 is further characterized by an XRPD pattern when measured using CuK_{α} radiation comprising one or more additional characteristic peaks, expressed in degrees 2-theta (±0.2), selected from 10.2 and 13.2. In some embodiments, the crystalline Form II of Compound 1 is characterized by an XRPD pattern when measured using CuK_{α} radiation comprising only four peaks, expressed in degrees 2-theta, from about 7 to about 12.

In some embodiments, the crystalline Form II of Compound 1 is characterized by an XRPD pattern when measured using CuK_{α} radiation that does not exhibit a peak at 4.3 degrees 2-theta (±0.2). In some embodiments, the crystalline Form II of Compound 1 is characterized by an XRPD pattern when measured using CuK_{α} radiation that does not exhibit a peak at 5.6 degrees 2-theta (±0.2).

In some embodiments, the crystalline Form II of Compound 1 is characterized by an XRPD pattern when measured using CuK_{α} radiation comprising an absence of peaks, expressed in degrees 2-theta, from about 3 to about 4.8. In some embodiments, the crystalline Form II of Compound 1 is characterized by an XRPD pattern when measured using CuK_{α} radiation comprising an absence of peaks, expressed in degrees 2-theta, from about 4.8 to about 6. In some embodiments, the crystalline Form II of Compound 1 is characterized by an XRPD pattern when measured using CuK_{α} radiation comprising an absence of peaks, expressed in degrees 2-theta, from about 3 to about 7. In some embodiments, the crystalline Form II of Compound 1 is characterized by an XRPD pattern when measured using CuK_{α} radiation comprising an absence of peaks, expressed in degrees 2-theta, from about 3 to about 7.5. In some embodiments, the crystalline Form II of Compound 1 is characterized by an XRPD pattern when measured using CuK_{α} radiation comprising an absence of peaks, expressed in degrees 2-theta, from about 11.2 to about 12.5.

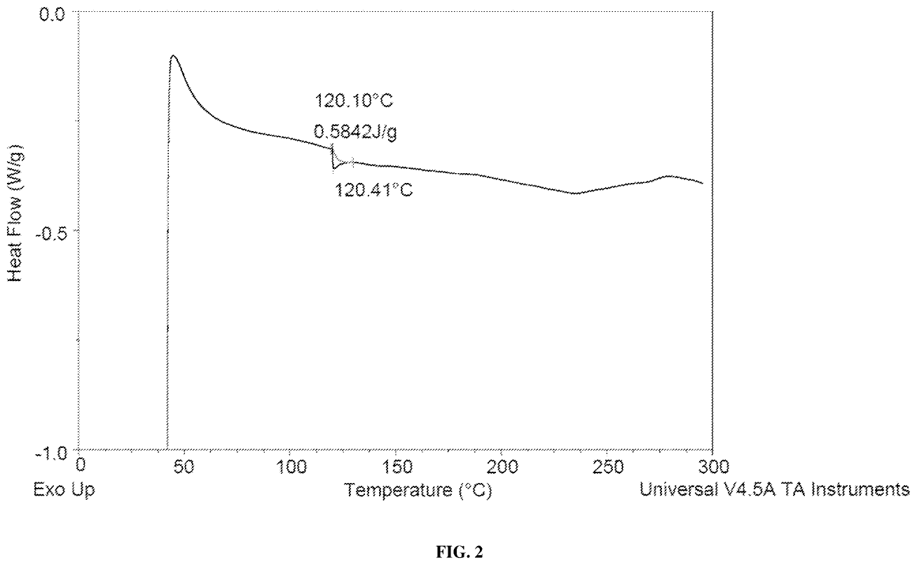

In some embodiments, the crystalline Form II of Compound 1 is characterized by a differential scanning calorimetry (DSC) thermogram substantially similar to that depicted in FIG. 2. In some embodiments, the crystalline Form II of Compound 1 is characterized by a DSC thermogram comprising an endotherm comprising an onset temperature (Tₘ) of about 120.1±2.0° C. In some embodiments, the crystalline Form II of Compound 1 is characterized by a DSC thermogram comprising a peak temperature (Tₘₐₓ) of about 120.41° C. In some embodiments, the crystalline Form II of Compound 1 is characterized by a DSC thermogram comprising an enthalpy of the endothermic transition (AH) of about 0.584±0.03 J/g.

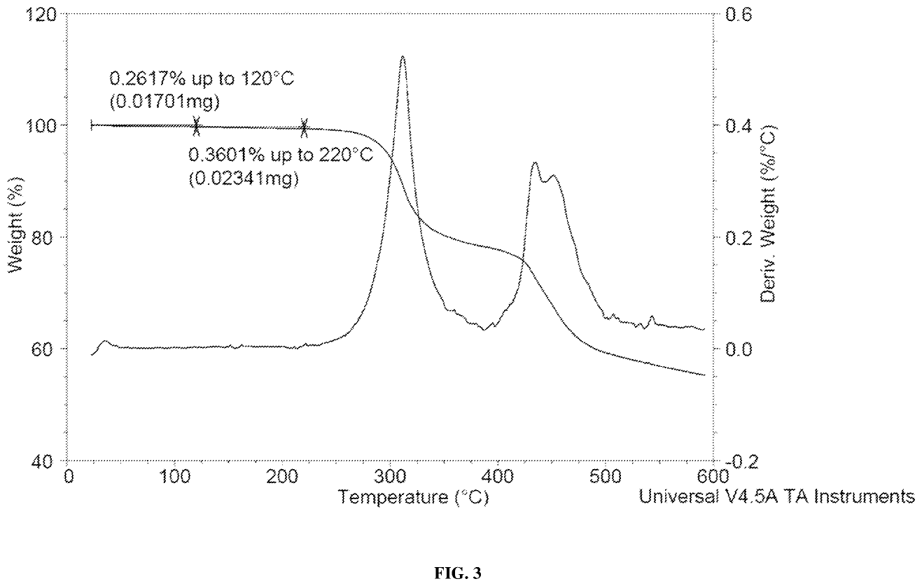

In some embodiments, the crystalline Form II of Compound 1 is characterized by a thermogravimetric analysis (TGA) thermogram substantially similar to that depicted in FIG. 3. In some embodiments, the crystalline Form II of Compound 1 is characterized by a TGA thermogram comprising a weight loss of about (0.262±0.013)% up to 120° C. In some embodiments, the crystalline Form II of Compound 1 is characterized by a TGA thermogram comprising a weight loss of about (0.360±0.018)% up to 220° C.

In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 25±5° C. or about 50±5° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 20° C. to about 55° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 20° C. to about 50° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 20° C. to about 45° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 25° C. to about 55° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 25° C. to about 50° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 25° C. to about 45° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 30° C. to about 55° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 30° C. to about 50° C. In some embodiments, the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 30° C. to about 45° C.

In some embodiments, the crystalline Form II of Compound 1 is obtained by phase equilibration in dichloromethane and methyl ethyl ketone at about 50±5° C. In some embodiments, the crystalline Form II of Compound 1 is obtained by phase equilibration in dichloromethane and methyl ethyl ketone at about 50±2° C. In some embodiments, the crystalline Form II of Compound 1 is obtained by phase equilibration in dichloromethane and methyl ethyl ketone at about 50° C. In some embodiments, the crystalline Form II of Compound 1 is obtained by phase equilibration in dichloromethane and methyl ethyl ketone at about 45° C. to about 55° C. In some embodiments, the crystalline Form II of Compound 1 is obtained by phase equilibration in dichloromethane and methyl ethyl ketone at about 45° C. to about 50° C. In some embodiments, the crystalline Form II of Compound 1 is obtained by phase equilibration in dichloromethane and methyl ethyl ketone at about 50° C. to about 55° C. In some embodiments, the crystalline Form II of Compound 1 is obtained by phase equilibration in dichloromethane and methyl ethyl ketone at about 48° C. to about 52° C. In some embodiments, the crystalline Form II of Compound 1 is obtained by phase equilibration in dichloromethane and methyl ethyl ketone at about 48° C. to about 50° C. In some embodiments, the crystalline Form II of Compound 1 is obtained by phase equilibration in dichloromethane and methyl ethyl ketone at about 50° C. to about 52° C.

In some embodiments, the crystalline Form II of Compound 1 is substantially free of impurities. In some embodiments, the crystalline Form II of Compound 1 is substantially free of other salts of Compound 1. In some embodiments, the crystalline Form II of Compound 1 is substantially free of solvents.

In some embodiments, the crystalline Form II of Compound 1 is substantially free of amorphous forms of Compound 1. In some embodiments, the crystalline Form II of Compound 1 is substantially free of amorphous forms of a salt of Compound 1. In some embodiments, the crystalline Form II of Compound 1 is substantially free of amorphous forms of a solvate of Compound 1. In some embodiments, the crystalline Form II of Compound 1 is substantially free of other crystalline forms of Compound 1.

In another aspect, the present disclosure provides a composition comprising the crystalline Form II of Compound 1, wherein the composition comprises greater than or equal to 90% by weight of the crystalline Form II of Compound 1.

In some embodiments, a composition comprising the crystalline Form II of Compound 1 comprises greater than or equal to 90% by weight of the crystalline Form II of Compound 1. In some embodiments, a composition comprising the crystalline Form II of Compound 1 comprises greater than or equal to 95% by weight of the crystalline Form II of Compound 1. In some embodiments, a composition comprising the crystalline Form II of Compound 1 comprises greater than or equal to 96% by weight of the crystalline Form II of Compound 1. In some embodiments, a composition comprising the crystalline Form II of Compound 1 comprises greater than or equal to 97% by weight of the crystalline Form II of Compound 1. In some embodiments, a composition comprising the crystalline Form II of Compound 1 comprises greater than or equal to 98% by weight of the crystalline Form II of Compound 1. In some embodiments, a composition comprising the crystalline Form II of Compound 1 comprises greater than or equal to 99% by weight of the crystalline Form II of Compound 1. In some embodiments, a composition comprising the crystalline Form II of Compound 1 comprises greater than or equal to 99.5% by weight of the crystalline Form II of Compound 1. In some embodiments, a composition comprising the crystalline Form II of Compound 1 comprises greater than or equal to 99.9% by weight of the crystalline Form II of Compound 1.

In another aspect, the present disclosure provides a composition comprising the crystalline Form II of Compound 1, wherein the molar ratio of the amount of Form II of Compound 1 to the sum of amounts of other forms in the composition is equal to or greater than 90:10.

In some embodiments, in a composition comprising the crystalline Form II of Compound 1, the molar ratio of the amount of Form II of Compound 1 to the sum of amounts of the other forms in the composition is equal to or greater than 95:5. In some embodiments, in a composition comprising the crystalline Form II of Compound 1, the molar ratio of the amount of Form II of Compound 1 to the sum of amounts of the other forms in the composition is equal to or greater than 96:4. In some embodiments, in a composition comprising the crystalline Form II of Compound 1, the molar ratio of the amount of Form II of Compound 1 to the sum of amounts of the other forms in the composition is equal to or greater than 97:3. In some embodiments, in a composition comprising the crystalline Form II of Compound 1, the molar ratio of the amount of Form II of Compound 1 to the sum of amounts of the other forms in the composition is equal to or greater than 98:2. In some embodiments, in a composition comprising the crystalline Form II of Compound 1, the molar ratio of the amount of Form II of Compound 1 to the sum of amounts of the other forms in the composition is equal to or greater than 99:1. In some embodiments, in a composition comprising the crystalline Form II of Compound 1, the molar ratio of the amount of Form II of Compound 1 to the sum of amounts of the other forms in the composition is equal to or greater than 99.5:0.5. In some embodiments, in a composition comprising the crystalline Form II of Compound 1, the molar ratio of the amount of Form II of Compound 1 to the sum of amounts of the other forms in the composition is equal to or greater than 99.9:0.1.

### Crystalline Form III of Compound 1

In another aspect, the present disclosure provides a crystalline Form III of Compound 1:

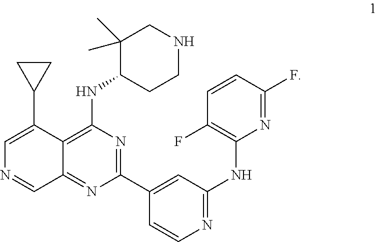 [SMILES: CC1(C)CNCC[C@@H]1Nc1nc(-c2ccnc(Nc3nc(F)ccc3F)c2)nc2cncc(C3CC3)c12]

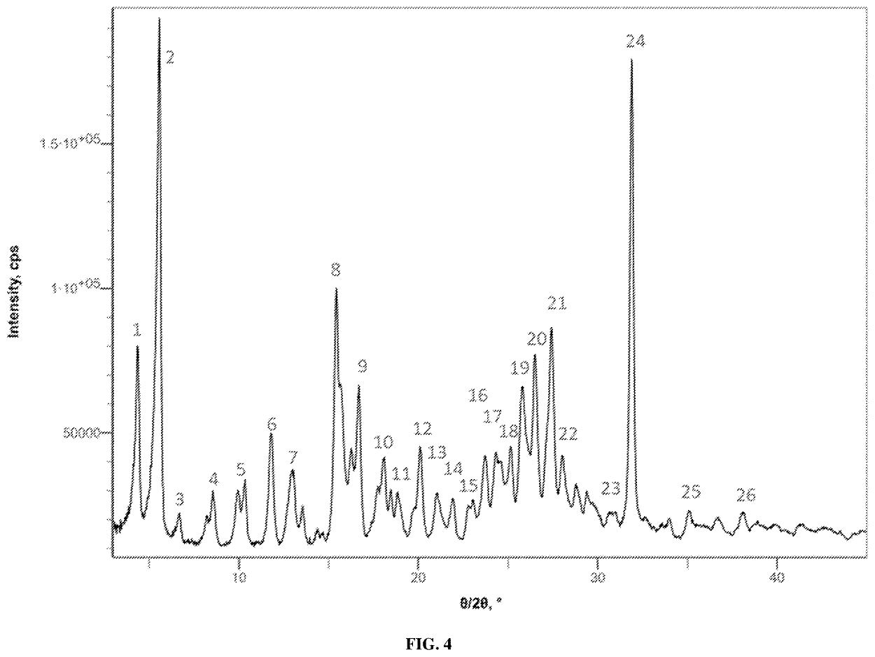

In some embodiments, the crystalline Form III of Compound 1 is characterized by an X-ray powder diffraction (XRPD) pattern substantially similar to that depicted in FIG. 4 when measured using CuK_{α} radiation. In some embodiments, the crystalline Form III of Compound 1 is characterized by an X-ray powder diffraction (XRPD) pattern substantially similar to that tabulated in Table 4.

In some embodiments, the crystalline Form III of Compound 1 is characterized by an XRPD pattern comprising three or more characteristic peaks, expressed in degrees 2-theta (±0.2), of 4.3, 5.6, and 11.8 when measured using CuK_{α} radiation. In some embodiments, the crystalline Form III of Compound 1 is characterized by an XRPD pattern comprising an additional characteristic peak, expressed in degrees 2-theta (±0.2), of 6.7 when measured using CuK_{α} radiation.

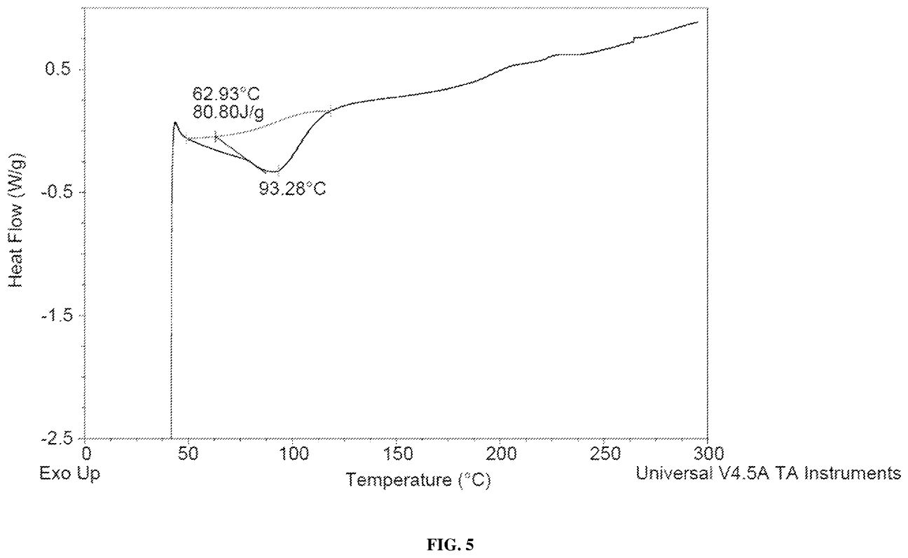

In some embodiments, the crystalline Form III of Compound 1 is characterized by a differential scanning calorimetry (DSC) thermogram substantially similar to that depicted in FIG. 5. In some embodiments, the crystalline Form III of Compound 1 is characterized by a DSC thermogram comprising an endotherm comprising an onset temperature (Tₘ) of about 62.93±2.0° C. In some embodiments, the crystalline Form III of Compound 1 is characterized by a DSC thermogram comprising a peak temperature (Tₘₐₓ) of about 93.28±2.0° C. In some embodiments, the crystalline Form III of Compound 1 is characterized by a DSC thermogram comprising an enthalpy of the endothermic transition (AH) of about 80.80±4.0 J/g.

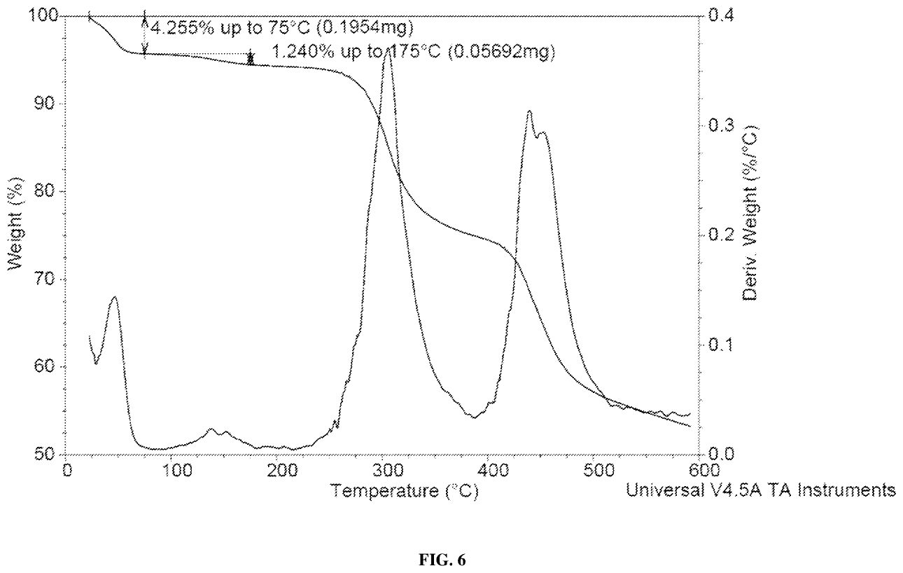

In some embodiments, the crystalline Form III of Compound 1 is characterized by a thermogravimetric analysis (TGA) thermogram substantially similar to that depicted in FIG. 6. In some embodiments, the crystalline Form III of Compound 1 is characterized by a TGA thermogram comprising a weight loss of about (4.255±0.2)% up to 75° C. In some embodiments, the crystalline Form III of Compound 1 is characterized by a TGA thermogram comprising a weight loss of about (1.240±0.060)% from 75° C. up to 175° C.

In some embodiments, the crystalline Form III of Compound 1 is obtained by phase equilibration in 1,4-dioxane at about 50±5° C. In some embodiments, the crystalline Form III of Compound 1 is obtained by phase equilibration in 1,4-dioxane at about 50±2° C. In some embodiments, the crystalline Form III of Compound 1 is obtained by phase equilibration in 1,4-dioxane at about 50° C. In some embodiments, the crystalline Form III of Compound 1 is obtained by phase equilibration in 1,4-dioxane at about 45° C. to about 55° C. In some embodiments, the crystalline Form III of Compound 1 is obtained by phase equilibration in 1,4-dioxane at about 45° C. to about 50° C. In some embodiments, the crystalline Form III of Compound 1 is obtained by phase equilibration in 1,4-dioxane at about 50° C. to about 55° C. In some embodiments, the crystalline Form III of Compound 1 is obtained by phase equilibration in 1,4-dioxane at about 48° C. to about 52° C. In some embodiments, the crystalline Form III of Compound 1 is obtained by phase equilibration in 1,4-dioxane at about 48° C. to about 50° C. In some embodiments, the crystalline Form III of Compound 1 is obtained by phase equilibration in 1,4-dioxane at about 50° C. to about 52° C.

In some embodiments, the crystalline Form III of Compound 1 is substantially free of impurities. In some embodiments, the crystalline Form III of Compound 1 is substantially free of other salts of Compound 1. In some embodiments, the crystalline Form III of Compound 1 is substantially free of solvents.

In some embodiments, the crystalline Form III of Compound 1 is substantially free of amorphous forms of Compound 1. In some embodiments, the crystalline Form III of Compound 1 is substantially free of amorphous forms of a salt of Compound 1. In some embodiments, the crystalline Form III of Compound 1 is substantially free of amorphous forms of a solvate of Compound 1. In some embodiments, the crystalline Form III of Compound 1 is substantially free of other crystalline forms of Compound 1.

In another aspect, the present disclosure provides a composition comprising the crystalline Form III of Compound 1, wherein the composition comprises greater than or equal to 90% by weight of the crystalline Form III of Compound 1.

In some embodiments, a composition comprising the crystalline Form III of Compound 1 comprises greater than or equal to 90% by weight of the crystalline Form III of Compound 1. In some embodiments, a composition comprising the crystalline Form III of Compound 1 comprises greater than or equal to 95% by weight of the crystalline Form III of Compound 1. In some embodiments, a composition comprising the crystalline Form III of Compound 1 comprises greater than or equal to 96% by weight of the crystalline Form III of Compound 1. In some embodiments, a composition comprising the crystalline Form III of Compound 1 comprises greater than or equal to 97% by weight of the crystalline Form III of Compound 1. In some embodiments, a composition comprising the crystalline Form III of Compound 1 comprises greater than or equal to 98% by weight of the crystalline Form III of Compound 1. In some embodiments, a composition comprising the crystalline Form III of Compound 1 comprises greater than or equal to 99% by weight of the crystalline Form III of Compound 1. In some embodiments, a composition comprising the crystalline Form III of Compound 1 comprises greater than or equal to 99.5% by weight of the crystalline Form III of Compound 1. In some embodiments, a composition comprising the crystalline Form III of Compound 1 comprises greater than or equal to 99.9% by weight of the crystalline Form III of Compound 1.

In another aspect, the present disclosure provides a composition comprising the crystalline Form III of Compound 1, wherein the molar ratio of the amount of Form III of Compound 1 to the sum of amounts of other forms in the composition is equal to or greater than 90:10.

In some embodiments, in a composition comprising the crystalline Form III of Compound 1, the molar ratio of the amount of Form III of Compound 1 to the sum of amounts of the other forms in the composition is equal to or greater than 95:5. In some embodiments, in a composition comprising the crystalline Form III of Compound 1, the molar ratio of the amount of Form III of Compound 1 to the sum of amounts of the other forms in the composition is equal to or greater than 96:4. In some embodiments, in a composition comprising the crystalline Form III of Compound 1, the molar ratio of the amount of Form III of Compound 1 to the sum of amounts of the other forms in the composition is equal to or greater than 97:3. In some embodiments, in a composition comprising the crystalline Form III of Compound 1, the molar ratio of the amount of Form III of Compound 1 to the sum of amounts of the other forms in the composition is equal to or greater than 98:2. In some embodiments, in a composition comprising the crystalline Form III of Compound 1, the molar ratio of the amount of Form III of Compound 1 to the sum of amounts of the other forms in the composition is equal to or greater than 99:1. In some embodiments, in a composition comprising the crystalline Form III of Compound 1, the molar ratio of the amount of Form III of Compound 1 to the sum of amounts of the other forms in the composition is equal to or greater than 99.5:0.5. In some embodiments, in a composition comprising the crystalline Form III of Compound 1, the molar ratio of the amount of Form III of Compound 1 to the sum of amounts of the other forms in the composition is equal to or greater than 99.9:0.1.

### Pharmaceutical Compositions, Kits, and Administration

In another aspect, the present disclosure provides a pharmaceutical composition comprising a crystalline form provided herein, or a composition provide herein, and a pharmaceutically acceptable excipient.

In another aspect, the present disclosure provides a kit comprising a crystalline form provided herein, a composition thereof, or a pharmaceutical composition thereof, and instructions for using the crystalline form, composition, or pharmaceutical composition.

Pharmaceutical compositions disclosed herein can be prepared by any method known in the art of pharmaceutics. In general, such preparatory methods include bringing the compound disclosed herein (i.e., the “active ingredient”) into association with a carrier or excipient, and/or one or more other accessory ingredients, and then, if necessary and/or desirable, shaping, and/or packaging the product into a desired single- or multi-dose unit.

In some embodiments, pharmaceutical compositions are prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition disclosed herein will vary, depending upon the identity, size, and/or condition of the subject treated and further depending upon the route by which the composition is to be administered. In some embodiments, the composition comprises between 0.1% and 100% (w/w) active ingredient.

Pharmaceutically acceptable excipients used in the manufacture of the disclosed pharmaceutical compositions include inert diluents, dispersing and/or granulating agents, surface active agents and/or emulsifiers, disintegrating agents, binding agents, preservatives, buffering agents, lubricating agents, and/or oils. In some embodiments, excipients such as cocoa butter and suppository waxes, coloring agents, coating agents, sweetening, flavoring, and perfuming agents are present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj© 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij© 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip©, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In some embodiments, dosage forms for topical and/or transdermal administration of a compound disclosed herein include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as required. Additionally, the present disclosure contemplates the use of transdermal patches, which often have the added advantage of providing controlled delivery of an active ingredient to the body. In some embodiments, such dosage forms are prepared, for example, by dissolving and/or dispensing the active ingredient in the proper medium. In some embodiments, alternatively or additionally, the rate is controlled by either providing a rate controlling membrane and/or by dispersing the active ingredient in a polymer matrix and/or gel. In some embodiments, the pharmaceutical composition is in the form of a topical formulation.

Suitable devices for use in delivering intradermal pharmaceutical compositions disclosed herein include short needle devices. In some embodiments, intradermal compositions are administered by devices which limit the effective penetration length of a needle into the skin. In some embodiments, alternatively or additionally, conventional syringes are used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. In some embodiments, topically administrable formulations, for example, comprise from about 1% to about 10% (w/w) active ingredient. In some embodiments, the concentration of the active ingredient is as high as the solubility limit of the active ingredient in the solvent. In some embodiments, formulations for topical administration further comprise one or more of the additional ingredients disclosed herein.

Although the descriptions of pharmaceutical compositions disclosed herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds disclosed herein are typically formulated in dosage unit form for ease of administration and uniformity of dosage. It will be understood, however, that the total daily usage of the compositions disclosed herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound, mode of administration, and the like. In some embodiments, an effective amount is included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound disclosed herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Dose ranges as disclosed herein provide guidance for the administration of the disclosed pharmaceutical compositions to an adult. In some embodiments, the amount to be administered to, for example, a child or an adolescent is determined by a medical practitioner or person skilled in the art and is lower or the same as that administered to an adult.

In some embodiments, the pharmaceutical composition comprises about 0.5 mg to about 1,500 mg of the compound of Compound 1, or a pharmaceutically acceptable salt thereof. In some embodiments, the pharmaceutical composition comprises about 0.5 mg to about 1,000 mg of the compound of Compound 1, or a pharmaceutically acceptable salt thereof. In some embodiments, the pharmaceutical composition comprises about 0.5 mg to about 500 mg of the compound of Compound 1, or a pharmaceutically acceptable salt thereof. In some embodiments, the pharmaceutical composition comprises about 0.5 mg to about 300 mg of the compound of Compound 1, or a pharmaceutically acceptable salt thereof. In some embodiments, the pharmaceutical composition comprises about 0.5 mg to about 250 mg of the compound of Compound 1, or a pharmaceutically acceptable salt thereof. In some embodiments, the pharmaceutical composition comprises about 0.5 mg to about 200 mg of the compound of Compound 1, or a pharmaceutically acceptable salt thereof. In some embodiments, the pharmaceutical composition comprises about 0.5 mg to about 150 mg of the compound of Compound 1, or a pharmaceutically acceptable salt thereof. In some embodiments, the pharmaceutical composition comprises about 0.5 mg to about 100 mg of the compound of Compound 1, or a pharmaceutically acceptable salt thereof. In some embodiments, the pharmaceutical composition comprises about 0.5 mg to about 50 mg of the compound of Compound 1, or a pharmaceutically acceptable salt thereof. In some embodiments, the pharmaceutical composition comprises about 0.5 mg to about 25 mg of the compound of Compound 1, or a pharmaceutically acceptable salt thereof. In some embodiments, the pharmaceutical composition comprises about 0.5 mg to about 20 mg of the compound of Compound 1, or a pharmaceutically acceptable salt thereof. In some embodiments, the pharmaceutical composition comprises about 0.5 mg to about 15 mg of the compound of Compound 1, or a pharmaceutically acceptable salt thereof. In some embodiments, the pharmaceutical composition comprises about 0.5 mg to about 10 mg of the compound of Compound 1, or a pharmaceutically acceptable salt thereof. In some embodiments, the pharmaceutical composition comprises about 1 mg to about 10 mg of the compound of Compound 1, or a pharmaceutically acceptable salt thereof. In some embodiments, the pharmaceutical composition comprises about 2 mg to about 8 mg of the compound of Compound 1, or a pharmaceutically acceptable salt thereof. In some embodiments, the pharmaceutical composition comprises about 4 mg to about 6 mg of the compound of Compound 1, or a pharmaceutically acceptable salt thereof.

In some embodiments, the pharmaceutical composition comprises about 0.5 mg, 1 mg, 1.5 mg, 2 mg, 3 mg, 4 mg, 5 mg, 6 mg, 7 mg, 8 mg, 9 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 110 mg, 120 mg, 140 mg, 160 mg, 180 mg, 200 mg, 220 mg, 240 mg, 260 mg, 280 mg, 300 mg, 400 mg, 500 mg, 600 mg, 700 mg, 800 mg, 900 mg, 1,000 mg, 1,250 mg, 1,500 mg of the compound of Compound 1, or a pharmaceutically acceptable salt thereof. In some embodiments, the pharmaceutical composition comprises about 1 mg of the compound of Compound 1, or a pharmaceutically acceptable salt thereof. In some embodiments, the pharmaceutical composition comprises about 2 mg of the compound of Compound 1, or a pharmaceutically acceptable salt thereof. In some embodiments, the pharmaceutical composition comprises about 3 mg of the compound of Compound 1, or a pharmaceutically acceptable salt thereof. In some embodiments, the pharmaceutical composition comprises about 4 mg of the compound of Compound 1, or a pharmaceutically acceptable salt thereof. In some embodiments, the pharmaceutical composition comprises about 5 mg of the compound of Compound 1, or a pharmaceutically acceptable salt thereof. In some embodiments, the pharmaceutical composition comprises about 6 mg of the compound of Compound 1, or a pharmaceutically acceptable salt thereof. In some embodiments, the pharmaceutical composition comprises about 7 mg of the compound of Compound 1, or a pharmaceutically acceptable salt thereof. In some embodiments, the pharmaceutical composition comprises about 8 mg of the compound of Compound 1, or a pharmaceutically acceptable salt thereof. In some embodiments, the pharmaceutical composition comprises about 9 mg of the compound of Compound 1, or a pharmaceutically acceptable salt thereof. In some embodiments, the pharmaceutical composition comprises about 10 mg of the compound of Compound 1, or a pharmaceutically acceptable salt thereof. In some embodiments, the pharmaceutical composition comprises about 12.5 mg of the compound of Compound 1, or a pharmaceutically acceptable salt thereof. In some embodiments, the pharmaceutical composition comprises about 15 mg of the compound of Compound 1, or a pharmaceutically acceptable salt thereof. In some embodiments, the pharmaceutical composition comprises about 20 mg of the compound of Compound 1, or a pharmaceutically acceptable salt thereof. In some embodiments, the pharmaceutical composition comprises about 25 mg of the compound of Compound 1, or a pharmaceutically acceptable salt thereof. In some embodiments, the pharmaceutical composition comprises about 30 mg of the compound of Compound 1, or a pharmaceutically acceptable salt thereof. In some embodiments, the pharmaceutical composition comprises about 35 mg of the compound of Compound 1, or a pharmaceutically acceptable salt thereof. In some embodiments, the pharmaceutical composition comprises about 40 mg of the compound of Compound 1, or a pharmaceutically acceptable salt thereof. In some embodiments, the pharmaceutical composition comprises about 45 mg of the compound of Compound 1, or a pharmaceutically acceptable salt thereof. In some embodiments, the pharmaceutical composition comprises about 50 mg of the compound of Compound 1, or a pharmaceutically acceptable salt thereof. In some embodiments, the pharmaceutical composition comprises about 55 mg of the compound of Compound 1, or a pharmaceutically acceptable salt thereof. In some embodiments, the pharmaceutical composition comprises about 60 mg of the compound of Compound 1, or a pharmaceutically acceptable salt thereof. In some embodiments, the pharmaceutical composition comprises about 65 mg of the compound of Compound 1, or a pharmaceutically acceptable salt thereof. In some embodiments, the pharmaceutical composition comprises about 70 mg of the compound of Compound 1, or a pharmaceutically acceptable salt thereof. In some embodiments, the pharmaceutical composition comprises about 75 mg of the compound of Compound 1, or a pharmaceutically acceptable salt thereof. In some embodiments, the pharmaceutical composition comprises about 80 mg of the compound of Compound 1, or a pharmaceutically acceptable salt thereof. In some embodiments, the pharmaceutical composition comprises about 85 mg of the compound of Compound 1, or a pharmaceutically acceptable salt thereof. In some embodiments, the pharmaceutical composition comprises about 90 mg of the compound of Compound 1, or a pharmaceutically acceptable salt thereof. In some embodiments, the pharmaceutical composition comprises about 100 mg of the compound of Compound 1, or a pharmaceutically acceptable salt thereof. In some embodiments, the pharmaceutical composition comprises about 110 mg of the compound of Compound 1, or a pharmaceutically acceptable salt thereof. In some embodiments, the pharmaceutical composition comprises about 120 mg of the compound of Compound 1, or a pharmaceutically acceptable salt thereof. In some embodiments, the pharmaceutical composition comprises about 140 mg of the compound of Compound 1, or a pharmaceutically acceptable salt thereof. In some embodiments, the pharmaceutical composition comprises about 160 mg of the compound of Compound 1, or a pharmaceutically acceptable salt thereof. In some embodiments, the pharmaceutical composition comprises about 180 mg of the compound of Compound 1, or a pharmaceutically acceptable salt thereof. In some embodiments, the pharmaceutical composition comprises about 200 mg of the compound of Compound 1, or a pharmaceutically acceptable salt thereof. In some embodiments, the pharmaceutical composition comprises about 220 mg of the compound of Compound 1, or a pharmaceutically acceptable salt thereof. In some embodiments, the pharmaceutical composition comprises about 240 mg of the compound of Compound 1, or a pharmaceutically acceptable salt thereof. In some embodiments, the pharmaceutical composition comprises about 260 mg of the compound of Compound 1, or a pharmaceutically acceptable salt thereof. In some embodiments, the pharmaceutical composition comprises about 280 mg of the compound of Compound 1, or a pharmaceutically acceptable salt thereof. In some embodiments, the pharmaceutical composition comprises about 300 mg of the compound of Compound 1, or a pharmaceutically acceptable salt thereof. In some embodiments, the pharmaceutical composition comprises about 400 mg of the compound of Compound 1, or a pharmaceutically acceptable salt thereof. In some embodiments, the pharmaceutical composition comprises about 500 mg of the compound of Compound 1, or a pharmaceutically acceptable salt thereof. In some embodiments, the pharmaceutical composition comprises about 600 mg of the compound of Compound 1, or a pharmaceutically acceptable salt thereof. In some embodiments, the pharmaceutical composition comprises about 700 mg of the compound of Compound 1, or a pharmaceutically acceptable salt thereof. In some embodiments, the pharmaceutical composition comprises about 800 mg of the compound of Compound 1, or a pharmaceutically acceptable salt thereof. In some embodiments, the pharmaceutical composition comprises about 900 mg of the compound of Compound 1, or a pharmaceutically acceptable salt thereof. In some embodiments, the pharmaceutical composition comprises about 1,000 mg of the compound of Compound 1, or a pharmaceutically acceptable salt thereof. In some embodiments, the pharmaceutical composition comprises about 1,250 mg of the compound of Compound 1, or a pharmaceutically acceptable salt thereof. In some embodiments, the pharmaceutical composition comprises about 1,500 mg of the compound of Compound 1, or a pharmaceutically acceptable salt thereof.

In some embodiments, a compound or composition, as disclosed herein, is administered in combination with one or more additional pharmaceutical agents (e.g., therapeutically and/or prophylactically active agents). In some embodiments, the compounds or compositions are administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of atypical protein kinase C in a subject or cell), improve bioavailability, improve safety, reduce drug resistance, reduce and/or modify metabolism, inhibit excretion, and/or modify distribution in a subject or cell. It will also be appreciated that, in some embodiments, the therapy employed achieves a desired effect for the same disorder and/or different effects. In certain embodiments, a pharmaceutical composition disclosed herein including a compound disclosed herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

In some embodiments, the compound or composition is administered concurrently with, prior to, or subsequent to one or more additional pharmaceutical agents. In some embodiments, the one or more additional pharmaceutical agents are useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In some embodiments, each additional pharmaceutical agent is administered at a dose and/or on a time schedule determined for that pharmaceutical agent. In some embodiments, the additional pharmaceutical agents are administered together with each other and/or with the compound or composition disclosed herein in a single dose or composition or administered separately in different doses or compositions. The particular combination to employ in a regimen will take into account compatibility of the compound disclosed herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Also encompassed by the disclosure are kits (e.g., pharmaceutical packs). In some embodiments, the kits disclosed herein comprise a pharmaceutical composition or compound disclosed herein and a container (e.g., a vial, ampule, bottle, syringe, and/or dispenser package, or other suitable container). In some embodiments, the disclosed kits optionally further include a second container comprising a pharmaceutical excipient for dilution or suspension of a pharmaceutical composition or compound disclosed herein. In some embodiments, the pharmaceutical composition or compound disclosed herein provided in the first container and the second container are combined to form one unit dosage form.

Thus, in one aspect, disclosed herein are kits including a first container comprising a compound or pharmaceutical composition disclosed herein. In certain embodiments, the kits are useful for treating BCC in a subject in need thereof. In certain embodiments, the kits are useful for preventing BCC in a subject in need thereof. In certain embodiments, the kits are useful for reducing the risk of BCC in a subject in need thereof. In certain embodiments, the kits are useful for inhibiting atypical protein kinase C in a subject or cell.

In certain embodiments, a kit disclosed herein further includes instructions for using the kit. In some embodiments, a kit disclosed herein also includes information as required by a regulatory agency such as the U.S. Food and Drug Administration (FDA). In certain embodiments, the information included in the kits is prescribing information. In certain embodiments, the kits and instructions provide for treating BCC in a subject in need thereof. In certain embodiments, the kits and instructions provide for preventing BCC in a subject in need thereof. In certain embodiments, the kits and instructions provide for reducing the risk of developing BCC in a subject in need thereof. In certain embodiments, the kits and instructions provide for inhibiting atypical protein kinase C in a subject or cell. In some embodiments, a kit disclosed herein includes one or more additional pharmaceutical agents disclosed herein as a separate composition.

### Methods of Preparation

In another aspect, the present disclosure provides a method of preparing a crystalline Form II of Compound 1, or a composition thereof, the method comprising the steps of:
(1) providing a solution of Compound 1 in one or more solvents;
(2) precipitating the crystalline Form II of Compound 1 from the solution of Compound 1; and
(3) isolating the crystalline Form II of Compound 1.

In some embodiments, the solution of Compound 1 is provided by heating a mixture of solid Compound 1 and one or more solvents. In some embodiments, at least one of the one or more solvents is a non-polar solvent. In some embodiments, at least one of the one or more solvents is a polar solvent. In some embodiments, at least one of the one or more solvents is an aprotic solvent. In some embodiments, at least one of the one or more solvents is a protic solvent. In some embodiments, at least one of the one or more solvents is acetonitrile, dichloromethane, dimethylformamide, 2-methoxy-ethanol, methyl iso-butyl ketone, acetone, n-butyl alcohol, methyl tert-butyl ether, ethyl acetate, heptane, isobutyl acetate, isopropyl acetate, methyl ethyl ketone, tetrahydrofuran, or dimethyl sulfoxide.

In some embodiments, the solution of Compound 1 is saturated or supersaturated. In some embodiments, the solution of Compound 1 is saturated. In some embodiments, the solution of Compound 1 is supersaturated.

In some embodiments, the precipitating step comprises addition of one or more seed crystals of crystalline Form II of Compound 1. In some embodiments, the precipitating step comprises one or more of phase equilibration, evaporation, anti-solvent addition, reverse addition, and cooling of a saturated solution. In some embodiments, the precipitating step comprises evaporation. In some embodiments, the precipitating step comprises anti-solvent addition. In some embodiments, the precipitating step comprises reverse addition. In some embodiments, the precipitating step comprises cooling of a saturated solution. In some embodiments, the precipitating step comprises phase equilibration.

In some embodiments, the precipitating step comprises phase equilibration in methyl iso-butyl acetone, n-butyl alcohol, methyl tert-butyl ether, ethyl acetate, iso-butyl acetate, iso-propyl acetate, or THF. In some embodiments, the precipitating step comprises phase equilibration in methyl iso-butyl acetone. In some embodiments, the precipitating step comprises phase equilibration in n-butyl alcohol. In some embodiments, the precipitating step comprises phase equilibration in methyl tert-butyl ether. In some embodiments, the precipitating step comprises phase equilibration in ethyl acetate. In some embodiments, the precipitating step comprises phase equilibration in iso-butyl acetate. In some embodiments, the precipitating step comprises phase equilibration in iso-propyl acetate. In some embodiments, the precipitating step comprises phase equilibration in THF.

In some embodiments, the phase equilibration is performed between about 25±5° C. and about 50±5° C. In some embodiments, the phase equilibration is performed between about 20° C. and about 55° C. In some embodiments, the phase equilibration is performed between about 20° C. and about 50° C. In some embodiments, the phase equilibration is performed between about 20° C. and about 45° C. In some embodiments, the phase equilibration is performed between about 25° C. and about 55° C. In some embodiments, the phase equilibration is performed between about 25° C. and about 50° C. In some embodiments, the phase equilibration is performed between about 25° C. and about 45° C. In some embodiments, the phase equilibration is performed between about 30° C. and about 55° C. In some embodiments, the phase equilibration is performed between about 30° C. and about 50° C. In some embodiments, the phase equilibration is performed between about 30° C. and about 45° C.

In some embodiments, the precipitating step comprises phase equilibration in dichloromethane or methyl ethyl ketone. In some embodiments, the precipitating step comprises phase equilibration in dichloromethane. In some embodiments, the precipitating step comprises phase equilibration in methyl ethyl ketone.

In some embodiments, the phase equilibration is performed at about 50±5° C. In some embodiments, the phase equilibration is performed at about 50±2° C. In some embodiments, the phase equilibration is performed at about 45° C. to about 55° C. In some embodiments, the phase equilibration is performed at about 45° C. to about 50° C. In some embodiments, the phase equilibration is performed at about 50° C. to about 55° C. In some embodiments, the phase equilibration is performed at about 48° C. to about 52° C. In some embodiments, the phase equilibration is performed at about 48° C. to about 50° C. In some embodiments, the phase equilibration is performed at about 50° C. to about 52° C. In some embodiments, the phase equilibration is performed at about 50° C.

In some embodiments, the precipitating step comprises anti-solvent addition to a solution of Compound 1 in dimethylformamide, 2-methoxy-ethanol, or dimethyl sulfoxide. In some embodiments, the precipitating step comprises anti-solvent addition to a solution of Compound 1 in dimethylformamide or 2-methoxy-ethanol. In some embodiments, the precipitating step comprises anti-solvent addition to a solution of Compound 1 in dimethylformamide or dimethyl sulfoxide. In some embodiments, the precipitating step comprises anti-solvent addition to a solution of Compound 1 in dimethylformamide or dimethyl sulfoxide. In some embodiments, the precipitating step comprises anti-solvent addition to a solution of Compound 1 in dimethylformamide. In some embodiments, the precipitating step comprises anti-solvent addition to a solution of Compound 1 in 2-methoxy-ethanol. In some embodiments, the precipitating step comprises anti-solvent addition to a solution of Compound 1 in dimethyl sulfoxide. In some embodiments, the anti-solvent is methyl tert-butyl ether or acetonitrile. In some embodiments, the anti-solvent is methyl tert-butyl ether. In some embodiments, the anti-solvent is acetonitrile.

In some embodiments, the precipitating step comprises reverse addition of an anti-solvent to a solution of Compound 1 in dimethylformamide, 2-methoxy-ethanol, or dimethyl sulfoxide. In some embodiments, the precipitating step comprises reverse addition of an anti-solvent to a solution of Compound 1 in dimethylformamide or 2-methoxy-ethanol. In some embodiments, the precipitating step comprises reverse addition of an anti-solvent to a solution of Compound 1 in dimethylformamide or dimethyl sulfoxide. In some embodiments, the precipitating step comprises reverse addition of an anti-solvent to a solution of Compound 1 in 2-methoxy-ethanol or dimethyl sulfoxide. In some embodiments, the precipitating step comprises reverse addition of an anti-solvent to a solution of Compound 1 in dimethylformamide. In some embodiments, the precipitating step comprises reverse addition of an anti-solvent to a solution of Compound 1 in 2-methoxy-ethanol. In some embodiments, the precipitating step comprises reverse addition of an anti-solvent to a solution of Compound 1 in dimethyl sulfoxide. In some embodiments, the anti-solvent is methyl tert-butyl ether or acetonitrile. In some embodiments, the anti-solvent is methyl tert-butyl ether. In some embodiments, the anti-solvent is acetonitrile.

In another aspect, the present disclosure provides a method of preparing a crystalline Form III of Compound 1, or a composition thereof, the method comprising the steps of:
(1) providing a solution of Compound 1 in one or more solvents;
(2) precipitating the crystalline Form III of Compound 1 from the solution of Compound 1; and
(3) isolating the crystalline Form III of Compound 1.

In some embodiments, the solution of Compound 1 is provided by heating a mixture of solid Compound 1 and one or more solvents. In some embodiments, at least one of the one or more solvents is a non-polar solvent. In some embodiments, at least one of the one or more solvents is a polar solvent. In some embodiments, at least one of the one or more solvents is an aprotic solvent. In some embodiments, at least one of the one or more solvents is a protic solvent. In some embodiments, at least one of the one or more solvents is acetonitrile, dichloromethane, dimethylformamide, 2-methoxy-ethanol, methyl iso-butyl ketone, acetone, n-butyl alcohol, methyl tert-butyl ether, ethyl acetate, heptane, isobutyl acetate, isopropyl acetate, methyl ethyl ketone, tetrahydrofuran, or dimethyl sulfoxide.

In some embodiments, at least one of the one or more solvents is 1,4-dioxane or n-propanol. In some embodiments, at least one of the one or more solvents is 1,4-dioxane. In some embodiments, at least one of the one or more solvents is n-propanol.

In some embodiments, the solution of Compound 1 is saturated or supersaturated. In some embodiments, the solution of Compound 1 is saturated. In some embodiments, the solution of Compound 1 is supersaturated.

In some embodiments, the precipitating step comprises addition of one or more seed crystals of crystalline Form III of Compound 1. In some embodiments, the precipitating step comprises one or more of phase equilibration, evaporation, anti-solvent addition, reverse addition, and cooling of a saturated solution. In some embodiments, the precipitating step comprises evaporation. In some embodiments, the precipitating step comprises anti-solvent addition. In some embodiments, the precipitating step comprises reverse addition. In some embodiments, the precipitating step comprises cooling of a saturated solution. In some embodiments, the precipitating step comprises phase equilibration.

In some embodiments, the precipitating step comprises phase equilibration in 1,4-dioxane.

In some embodiments, the phase equilibration is performed at about 50±5° C. In some embodiments, the phase equilibration is performed at about 50±2° C. In some embodiments, the phase equilibration is performed at about 45° C. to about 55° C. In some embodiments, the phase equilibration is performed at about 45° C. to about 50° C. In some embodiments, the phase equilibration is performed at about 50° C. to about 55° C. In some embodiments, the phase equilibration is performed at about 48° C. to about 52° C. In some embodiments, the phase equilibration is performed at about 48° C. to about 50° C. In some embodiments, the phase equilibration is performed at about 50° C. to about 52° C. In some embodiments, the phase equilibration is performed at about 50° C.

### Methods of Treatment and Uses

In another aspect, the present disclosure provides a method for treating cancer in a subject in need thereof, comprising administering to the subject a crystalline form provided herein, a composition thereof, or a pharmaceutical composition thereof.

In another aspect, the present disclosure provides a crystalline form provided herein, a composition thereof, or a pharmaceutical composition thereof for use in treating cancer in a subject in need thereof.

In another aspect, the present disclosure provides a use of a crystalline form provided herein, a composition thereof, or a pharmaceutical composition thereof for the treatment of cancer in a subject in need thereof.

In another aspect, the present disclosure provides a use of a crystalline form provided herein, a composition thereof, or a pharmaceutical composition thereof in the manufacture of a medicament for the treatment of basal cell carcinoma, solid tumors, or acute myeloid leukemia in a subject in need thereof.

In some embodiments, the cancer is basal cell carcinoma, a solid tumor, or acute myeloid leukemia. In some embodiments, the cancer is basal cell carcinoma or a solid tumor. In some embodiments, the cancer is basal cell carcinoma or acute myeloid leukemia. In some embodiments, the cancer is a solid tumor or acute myeloid leukemia. In some embodiments, the cancer is basal cell carcinoma. In some embodiments, the cancer is a solid tumor. In some embodiments, the cancer is acute myeloid leukemia.

In some embodiments, the topical formulation comprises a therapeutically effective amount of a compound of Compound 1. In some embodiments, the therapeutically effective amount is about 0.5 mg to about 1,500 mg of the compound of Compound 1, or a pharmaceutically acceptable salt thereof. In some embodiments, the therapeutically effective amount is about 0.5 mg to about 1,000 mg of the compound of Compound 1, or a pharmaceutically acceptable salt thereof. In some embodiments, the therapeutically effective amount is about 0.5 mg to about 500 mg of the compound of Compound 1, or a pharmaceutically acceptable salt thereof. In some embodiments, the therapeutically effective amount is about 0.5 mg to about 300 mg of the compound of Compound 1, or a pharmaceutically acceptable salt thereof. In some embodiments, the therapeutically effective amount is about 0.5 mg to about 250 mg of the compound of Compound 1, or a pharmaceutically acceptable salt thereof. In some embodiments, the therapeutically effective amount is about 0.5 mg to about 200 mg of the compound of Compound 1, or a pharmaceutically acceptable salt thereof. In some embodiments, the therapeutically effective amount is about 0.5 mg to about 150 mg of the compound of Compound 1, or a pharmaceutically acceptable salt thereof. In some embodiments, the therapeutically effective amount is about 0.5 mg to about 100 mg of the compound of Compound 1, or a pharmaceutically acceptable salt thereof. In some embodiments, the therapeutically effective amount is about 0.5 mg to about 50 mg of the compound of Compound 1, or a pharmaceutically acceptable salt thereof. In some embodiments, the therapeutically effective amount is about 0.5 mg to about 25 mg of the compound of Compound 1, or a pharmaceutically acceptable salt thereof. In some embodiments, the therapeutically effective amount is about 0.5 mg to about 20 mg of the compound of Compound 1, or a pharmaceutically acceptable salt thereof. In some embodiments, the therapeutically effective amount is about 0.5 mg to about 15 mg of the compound of Compound 1, or a pharmaceutically acceptable salt thereof. In some embodiments, the therapeutically effective amount is about 0.5 mg to about 10 mg of the compound of Compound 1, or a pharmaceutically acceptable salt thereof. In some embodiments, the therapeutically effective amount is about 1 mg to about 10 mg of the compound of Compound 1, or a pharmaceutically acceptable salt thereof. In some embodiments, the therapeutically effective amount is about 2 mg to about 8 mg of the compound of Compound 1, or a pharmaceutically acceptable salt thereof. In some embodiments, the therapeutically effective amount is about 4 mg to about 6 mg of the compound of Compound 1, or a pharmaceutically acceptable salt thereof.

In some embodiments, the therapeutically effective amount is about 0.5 mg, 1 mg, 2 mg, 3 mg, 4 mg, 5 mg, 6 mg, 7 mg, 8 mg, 9 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 110 mg, 120 mg, 140 mg, 160 mg, 180 mg, 200 mg, 220 mg, 240 mg, 260 mg, 280 mg, 300 mg, 400 mg, 500 mg, 600 mg, 700 mg, 800 mg, 900 mg, 1,000 mg, 1,250 mg, 1,500 mg of the compound of Compound 1, or a pharmaceutically acceptable salt thereof. In some embodiments, the therapeutically effective amount is about 0.5 mg of the compound of Compound 1, or a pharmaceutically acceptable salt thereof. In some embodiments, the therapeutically effective amount is about 1 mg of the compound of Compound 1, or a pharmaceutically acceptable salt thereof. In some embodiments, the therapeutically effective amount is about 2 mg of the compound of Compound 1, or a pharmaceutically acceptable salt thereof. In some embodiments, the therapeutically effective amount is about 3 mg of the compound of Compound 1, or a pharmaceutically acceptable salt thereof. In some embodiments, the therapeutically effective amount is about 4 mg of the compound of Compound 1, or a pharmaceutically acceptable salt thereof. In some embodiments, the therapeutically effective amount is about 5 mg of the compound of Compound 1, or a pharmaceutically acceptable salt thereof. In some embodiments, the therapeutically effective amount is about 6 mg of the compound of Compound 1, or a pharmaceutically acceptable salt thereof. In some embodiments, the therapeutically effective amount is about 7 mg of the compound of Compound 1, or a pharmaceutically acceptable salt thereof. In some embodiments, the therapeutically effective amount is about 8 mg of the compound of Compound 1, or a pharmaceutically acceptable salt thereof. In some embodiments, the therapeutically effective amount is about 9 mg of the compound of Compound 1, or a pharmaceutically acceptable salt thereof. In some embodiments, the therapeutically effective amount is about 10 mg of the compound of Compound 1, or a pharmaceutically acceptable salt thereof. In some embodiments, the therapeutically effective amount is about 12.5 mg of the compound of Compound 1, or a pharmaceutically acceptable salt thereof. In some embodiments, the therapeutically effective amount is about 15 mg of the compound of Compound 1, or a pharmaceutically acceptable salt thereof. In some embodiments, the therapeutically effective amount is about 20 mg of the compound of Compound 1, or a pharmaceutically acceptable salt thereof. In some embodiments, the therapeutically effective amount is about 25 mg of the compound of Compound 1, or a pharmaceutically acceptable salt thereof. In some embodiments, the therapeutically effective amount is about 30 mg of the compound of Compound 1, or a pharmaceutically acceptable salt thereof. In some embodiments, the therapeutically effective amount is about 35 mg of the compound of Compound 1, or a pharmaceutically acceptable salt thereof. In some embodiments, the therapeutically effective amount is about 40 mg of the compound of Compound 1, or a pharmaceutically acceptable salt thereof. In some embodiments, the therapeutically effective amount is about 45 mg of the compound of Compound 1, or a pharmaceutically acceptable salt thereof. In some embodiments, the therapeutically effective amount is about 50 mg of the compound of Compound 1, or a pharmaceutically acceptable salt thereof. In some embodiments, the therapeutically effective amount is about 55 mg of the compound of Compound 1, or a pharmaceutically acceptable salt thereof. In some embodiments, the therapeutically effective amount is about 60 mg of the compound of Compound 1, or a pharmaceutically acceptable salt thereof. In some embodiments, the therapeutically effective amount is about 65 mg of the compound of Compound 1, or a pharmaceutically acceptable salt thereof. In some embodiments, the therapeutically effective amount is about 70 mg of the compound of Compound 1, or a pharmaceutically acceptable salt thereof. In some embodiments, the therapeutically effective amount is about 75 mg of the compound of Compound 1, or a pharmaceutically acceptable salt thereof. In some embodiments, the therapeutically effective amount is about 80 mg of the compound of Compound 1, or a pharmaceutically acceptable salt thereof. In some embodiments, the therapeutically effective amount is about 85 mg of the compound of Compound 1, or a pharmaceutically acceptable salt thereof. In some embodiments, the therapeutically effective amount is about 90 mg of the compound of Compound 1, or a pharmaceutically acceptable salt thereof. In some embodiments, the therapeutically effective amount is about 100 mg of the compound of Compound 1, or a pharmaceutically acceptable salt thereof. In some embodiments, the therapeutically effective amount is about 110 mg of the compound of Compound 1, or a pharmaceutically acceptable salt thereof. In some embodiments, the therapeutically effective amount is about 120 mg of the compound of Compound 1, or a pharmaceutically acceptable salt thereof. In some embodiments, the therapeutically effective amount is about 140 mg of the compound of Compound 1, or a pharmaceutically acceptable salt thereof. In some embodiments, the therapeutically effective amount is about 160 mg of the compound of Compound 1, or a pharmaceutically acceptable salt thereof. In some embodiments, the therapeutically effective amount is about 180 mg of the compound of Compound 1, or a pharmaceutically acceptable salt thereof. In some embodiments, the therapeutically effective amount is about 200 mg of the compound of Compound 1, or a pharmaceutically acceptable salt thereof. In some embodiments, the therapeutically effective amount is about 220 mg of the compound of Compound 1, or a pharmaceutically acceptable salt thereof. In some embodiments, the therapeutically effective amount is about 240 mg of the compound of Compound 1, or a pharmaceutically acceptable salt thereof. In some embodiments, the therapeutically effective amount is about 260 mg of the compound of Compound 1, or a pharmaceutically acceptable salt thereof. In some embodiments, the therapeutically effective amount is about 280 mg of the compound of Compound 1, or a pharmaceutically acceptable salt thereof. In some embodiments, the therapeutically effective amount is about 300 mg of the compound of Compound 1, or a pharmaceutically acceptable salt thereof. In some embodiments, the therapeutically effective amount is about 400 mg of the compound of Compound 1, or a pharmaceutically acceptable salt thereof. In some embodiments, the therapeutically effective amount is about 500 mg of the compound of Compound 1, or a pharmaceutically acceptable salt thereof. In some embodiments, the therapeutically effective amount is about 600 mg of the compound of Compound 1, or a pharmaceutically acceptable salt thereof. In some embodiments, the therapeutically effective amount is about 700 mg of the compound of Compound 1, or a pharmaceutically acceptable salt thereof. In some embodiments, the therapeutically effective amount is about 800 mg of the compound of Compound 1, or a pharmaceutically acceptable salt thereof. In some embodiments, the therapeutically effective amount is about 900 mg of the compound of Compound 1, or a pharmaceutically acceptable salt thereof. In some embodiments, the therapeutically effective amount is about 1,000 mg of the compound of Compound 1, or a pharmaceutically acceptable salt thereof. In some embodiments, the therapeutically effective amount is about 1,250 mg of the compound of Compound 1, or a pharmaceutically acceptable salt thereof. In some embodiments, the therapeutically effective amount is about 1,500 mg of the compound of Compound 1, or a pharmaceutically acceptable salt thereof.

### EXAMPLES

In order that the present disclosure may be more fully understood, the following examples are set forth. The examples disclosed in this application are offered to illustrate the methods disclosed herein and are not to be construed in any way as limiting in their scope.

### Example 1. Phase Equilibration at 25±1° C. and 50±1° C.

Forms II and III of Compound 1 were prepared by phase equilibration from various solvents at 50±1° C. and 25±1° C. Compound 1 (20-100 mg) was added to solvent until a cloudy solution was obtained, then about 20 mg of Compound 1 was added to the cloudy solution. The mixture was stirred at 50±1° C. for 24 hours or at 25±1° C. for 48 hours. The solid was filtered and analyzed by XRPD.

Form II of Compound 1 was prepared by phase equilibration in methyl iso-butyl acetone, n-BuOH, MTBE, EtOA, iso-butyl acetate, iso-propyl acetate, and THF both at 25° C. and 50° C. Form II of Compound 1 was also prepared by phase equilibration in dichloromethane and methyl ethyl ketone at 50° C.

Form III of Compound 1 was prepared by phase equilibration in 1,4-dioxane at 50° C.

### Example 2. Anti-Solvent Addition

Form II of Compound 1 was prepared by anti-solvent addition. Saturated and nearly saturated solutions of Compound 1 (250 mg) were prepared in 2.5 mL of DMF, MOEtOH, or DMSO at room temperature. 15 mL of an anti-solvent (MTBE or MeCN) was added dropwise to induce precipitation of Form II of Compound 1 as shown in Table 1.

TABLE 1
Precipitation from anti-solvent addition.

| Solvent | Anti-solvent | Solid Form |
|---|---|---|
| DMF | MTBE | Form II |
| DMF | MeCN | Form II |
| MeOEtOH | MeCN | Form II |
| DMSO | MeCN | Form II |

### Example 3. Reverse Addition

Form II of Compound 1 was prepared by reverse addition. Saturated solutions and nearly saturated solutions of Compound 1 (250 mg) in 2.5 mL of DMF, MeOEtOH, DMSO, or ethyl formate were prepared at 25° C. and then added dropwise to a larger volume (15 mL) of a miscible anti-solvent (MeCN or MTBE) to afford Form II of Compound 1 as shown in Table 2.

TABLE 2
Precipitation from reverse addition.

| Solvent | Anti-solvent | Solid Form |
|---|---|---|
| DMF | MTBE | Form II |
| DMF | MeCN | Form II |
| MeOEtOH | MeCN | Form II |
| DMSO | MeCN | Form II |

### Example 4. Crystalline Form Characterization

Forms II and III of Compound 1 were characterized by XRPD (FIGS. 1 and 4, respectively), DSC (FIGS. 2 and 5, respectively), and TGA (FIGS. 3 and 6, respectively). X-Ray Powder Diffraction analysis was carried out on a Rigaku MiniFlex 6G X-ray Powder Diffractometer (XRPD) instrument. The general experimental procedures for XRPD were: (1) X-ray radiation from copper at 1.054056 Å with K_{b} filter; (2) X-ray power at 30 KV, 15 mA; and (3) the sample powder was dispersed on a zero-background sample holder. The general measurement conditions for XRPD were: start angle 3 degrees; stop angle 45 degrees; sampling 0.02 degrees; and scan speed 2 degree/min.

Differential Scanning Calorimetry (DSC) was carried out on a TA Instruments Differential Scanning Calorimetry, Model Q200 with autosampler. The DSC instrument conditions were as follows: 45° C.-300° C. at 10° C./min; Tzero aluminum sample pan and lid; and nitrogen gas flow at 50 mL/min. For Form II of Compound 1, 1.2000 mg of sample was analyzed. For Form III of Compound 1, 2.8000 mg was analyzed.

TGA thermograms were collected using a TA Instruments Thermogravimetric Analyzer, TGA Q500 with autosampler. The general experimental conditions for TGA were ramp from 25° C.-600° C. at 20° C./min; nitrogen purge, gas flow at 25 mL/min; platinum sample pan. For Form II of Compound 1, 6.5020 mg was analyzed. For Form III of Compound 1.

### Example 5. Tabulated XRPD Data for Form II

Table 3 provides a tabulated list corresponding to the labeled peaks in FIG. 1 of the XPRD pattern of Form II of Compound 1.

TABLE 3
XRPD Peaks of Form II

| Peak | | | Intensity, | | Height, |
|---|---|---|---|---|---|
| No. | LocationX | FWHMX | cps | Asymmetry | cps |
| 1 | 8.12721 | 0.24424 | 12226.18 | 1.85199 | 40507.35384 |
| 2 | 9.60484 | 0.26498 | 14714.94 | 2.97032 | 47744.05622 |
| 3 | 10.18599 | 0.24363 | 19149.42 | 2.97032 | 67575.44381 |
| 4 | 10.94160 | 0.24096 | 15538.80 | 1.90691 | 56189.54953 |
| 5 | 13.17042 | 0.33748 | 63889.55 | 2.75240 | 155148.24304 |
| 6 | 15.76659 | 0.27629 | 26807.51 | 2.45727 | 84573.22453 |
| 7 | 16.14186 | 0.51142 | 18719.07 | 2.45727 | 31903.63624 |
| 8 | 17.03638 | 0.25605 | 7470.28 | 1.91139 | 20426.29113 |
| 9 | 18.11926 | 0.28091 | 14783.98 | 1.38566 | 46617.25231 |
| 10 | 19.12148 | 0.18423 | 1460.00 | 1.27893 | 7388.09313 |
| 11 | 20.31087 | 0.72076 | 35336.85 | 0.55991 | 46054.68724 |
| 12 | 20.96170 | 0.35691 | 32722.81 | 0.55991 | 86124.81099 |
| 13 | 22.31582 | 0.22548 | 3449.83 | 1.29434 | 14373.08143 |
| 14 | 22.72121 | 0.23861 | 7528.27 | 1.29434 | 29640.27267 |
| 15 | 23.88752 | 0.26158 | 13372.39 | 1.49752 | 48025.34278 |
| 16 | 24.42228 | 0.26839 | 38889.87 | 1.49752 | 136126.77930 |
| 17 | 25.82801 | 0.31669 | 23513.06 | 1.74234 | 65638.31417 |
| 18 | 26.37109 | 0.20991 | 4940.74 | 1.74234 | 20808.42043 |
| 19 | 26.83545 | 0.18636 | 3284.02 | 1.74234 | 15579.20722 |
| 20 | 27.49152 | 0.22580 | 10409.98 | 1.74234 | 40758.58041 |
| 21 | 27.99163 | 0.24088 | 20917.52 | 1.74234 | 76770.39063 |
| 22 | 28.94306 | 0.18619 | 1113.06 | 0.95033 | 5132.69716 |
| 23 | 30.42118 | 0.51867 | 4714.14 | 0.63625 | 7714.84414 |
| 24 | 31.74949 | 0.26888 | 40637.81 | 1.15156 | 139225.03540 |
| 25 | 32.80000 | 0.28461 | 2562.40 | 0.85759 | 6146.11599 |
| 26 | 35.06643 | 0.60135 | 3574.84 | 0.61162 | 5584.64908 |
| 27 | 37.34303 | 0.34241 | 3688.28 | 1.39823 | 7700.23508 |
| 28 | 38.12363 | 0.27836 | 8415.91 | 1.39823 | 21613.39583 |
| 29 | 39.63387 | 0.29031 | 3338.39 | 1.01536 | 10803.02138 |
| 30 | 43.34250 | 0.45257 | 2158.64 | 0.81141 | 4336.89824 |
| 31 | 44.60881 | 0.38222 | 2141.21 | 1.92619 | 5259.75408 |

### Example 6. Tabulated XRPD Data for Form III of Compound 1

Table 4 provides a tabulated list corresponding to the labeled peaks in FIG. 4 of the XPRD pattern of Form III of Compound 1.

TABLE 4
XRPD Peaks of Form III of Compound 1

| Peak | | | Intensity, | | Height, |
|---|---|---|---|---|---|
| No. | LocationX | FWHMX | cps | Asymmetry | cps |
| 1 | 4.33867 | 0.22224 | 22355.08 | 0.91697 | 63401.73920 |
| 2 | 5.57814 | 0.29914 | 58096.94 | 2.28052 | 158843.16027 |
| 3 | 6.66449 | 0.16118 | 757.37 | 1.13561 | 4414.30038 |
| 4 | 8.53375 | 0.26906 | 7071.06 | 0.84908 | 16935.54568 |
| 5 | 10.03846 | 0.65164 | 11347.08 | 0.74328 | 16243.24667 |
| 6 | 11.79480 | 0.30697 | 12635.36 | 1.22652 | 37193.24291 |
| 7 | 13.01356 | 0.42753 | 11559.54 | 2.13910 | 21825.72150 |
| 8 | 15.42074 | 0.49532 | 51587.20 | 0.51138 | 66659.86917 |
| 9 | 16.67907 | 0.22126 | 11808.09 | 1.20882 | 37933.43403 |
| 10 | 18.10230 | 0.37441 | 8019.01 | 2.77847 | 16930.88513 |
| 11 | 18.84530 | 0.21298 | 2057.34 | 0.71516 | 7483.54788 |
| 12 | 20.09798 | 0.27222 | 7043.40 | 1.23555 | 24307.26530 |
| 13 | 21.06246 | 0.32263 | 2897.29 | 0.84896 | 8436.30005 |
| 14 | 21.88103 | 0.21289 | 1677.23 | 0.84896 | 7401.34024 |
| 15 | 22.95113 | 0.40030 | 2097.33 | 0.85535 | 4902.67146 |
| 16 | 23.67783 | 0.30798 | 5902.22 | 0.85535 | 17932.70464 |
| 17 | 24.43483 | 0.59231 | 9794.33 | 0.85535 | 15473.06117 |
| 18 | 25.11770 | 0.24086 | 4238.46 | 0.85535 | 16466.32708 |
| 19 | 25.82511 | 0.45664 | 14986.16 | 0.85535 | 30708.85559 |
| 20 | 26.47154 | 0.28553 | 12311.11 | 0.85535 | 40345.35791 |
| 21 | 27.42051 | 0.34707 | 18834.61 | 1.69257 | 50980.25009 |
| 22 | 28.05437 | 0.22524 | 3021.02 | 1.69257 | 12600.09177 |
| 23 | 30.80137 | 0.53941 | 629.91 | 0.50781 | 671.99583 |
| 24 | 31.88291 | 0.23655 | 45313.07 | 1.31317 | 159462.40155 |
| 25 | 35.05305 | 0.26071 | 1825.29 | 0.78434 | 5346.91714 |
| 26 | 38.06674 | 0.28935 | 1601.90 | 1.00022 | 5096.96411 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments disclosed herein. The scope of the present embodiments disclosed herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A crystalline Form II of Compound 1:

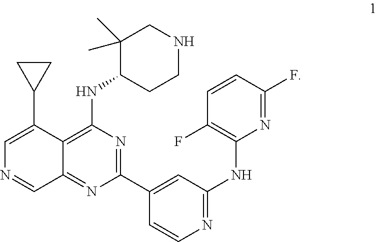 [SMILES: CC1(C)CNCC[C@@H]1Nc1nc(-c2ccnc(Nc3nc(F)ccc3F)c2)nc2cncc(C3CC3)c12]

2. The crystalline Form II of Compound 1 of claim 1, wherein the crystalline Form II of Compound 1 is characterized by an X-ray powder diffraction (XRPD) pattern substantially similar to that depicted in FIG. 1 when measured using CuK_{α} radiation.

3. The crystalline Form II of Compound 1 of claim 1 or 2, wherein the crystalline Form II of Compound 1 is characterized by an XRPD pattern when measured using CuK_{α} radiation comprising characteristic peaks, expressed in degrees 2-theta (±0.2), at 8.1, 9.6, and 10.9.

4. The crystalline Form II of Compound 1 of claim 3, wherein the crystalline Form II of Compound 1 is further characterized by an XRPD pattern when measured using CuK_{α} radiation comprising one or more additional characteristic peaks, expressed in degrees 2-theta (±0.2), selected from 10.2 and 13.2.

5. The crystalline Form II of Compound 1 of any one of claims 1-4, wherein the crystalline Form II of Compound 1 is characterized by an XRPD pattern when measured using CuK_{α} radiation that does not exhibit a peak at 4.3 degrees 2-theta (±0.2).

6. The crystalline Form II of Compound 1 of any one of claims 1-5, wherein the crystalline Form II of Compound 1 is characterized by an XRPD pattern when measured using CuK_{α} radiation that does not exhibit a peak at 5.6 degrees 2-theta (±0.2).

7. The crystalline Form II of Compound 1 of any one of claims 1-6, wherein the crystalline Form II of Compound 1 is characterized by an XRPD pattern when measured using CuK_{α} radiation comprising an absence of peaks, expressed in degrees 2-theta, from about 3 to about 4.8.

8. The crystalline Form II of Compound 1 of any one of claims 1-7, wherein the crystalline Form II of Compound 1 is characterized by an XRPD pattern when measured using CuK_{α} radiation comprising an absence of peaks, expressed in degrees 2-theta, from about 4.8 to about 6.

9. The crystalline Form II of Compound 1 of any one of claims 1-8, wherein the crystalline Form II of Compound 1 is characterized by an XRPD pattern when measured using CuK_{α} radiation comprising an absence of peaks, expressed in degrees 2-theta, from about 3 to about 7.

10. The crystalline Form II of Compound 1 of any one of claims 1-9, wherein the crystalline Form II of Compound 1 is characterized by an XRPD pattern when measured using CuK_{α} radiation comprising an absence of peaks, expressed in degrees 2-theta, from about 3 to about 7.5.

11. The crystalline Form II of Compound 1 of any one of claims 1-10, wherein the crystalline Form II of Compound 1 is characterized by an XRPD pattern when measured using CuK_{α} radiation comprising an absence of peaks, expressed in degrees 2-theta, from about 11.2 to about 12.5.

12. The crystalline Form II of Compound 1 of any one of claims 1-11, wherein the crystalline Form II of Compound 1 is characterized by an XRPD pattern when measured using CuK_{α} radiation comprising only four peaks, expressed in degrees 2-theta, from about 7 to about 12.

13. The crystalline Form II of Compound 1 of any one of claims 1-12, wherein the crystalline Form II of Compound 1 is characterized by a differential scanning calorimetry (DSC) thermogram substantially similar to that depicted in FIG. 2.

14. The crystalline Form II of Compound 1 of any one of claims 1-13, wherein the crystalline Form II of Compound 1 is characterized by a DSC thermogram comprising an endotherm comprising an onset temperature (Tₘ) of about 120.1±2.0° C.

15. The crystalline Form II of Compound 1 of any one of claims 1-14, wherein the crystalline Form II of Compound 1 is characterized by a DSC thermogram comprising a peak temperature (Tₘₐₓ) of about 120.41° C.

16. The crystalline Form II of Compound 1 of any one of claims 1-15, wherein the crystalline Form II of Compound 1 is characterized by a DSC thermogram comprising an enthalpy of the endothermic transition (AH) of about 0.584±0.03 J/g.

17. The crystalline Form II of Compound 1 of any one of claims 1-16, wherein the crystalline Form II of Compound 1 is characterized by a thermogravimetric analysis (TGA) thermogram substantially similar to that depicted in FIG. 3.

18. The crystalline Form II of Compound 1 of any one of claims 1-17, wherein the crystalline Form II of Compound 1 is characterized by a TGA thermogram comprising a weight loss of about (0.262±0.013)% up to 120° C.

19. The crystalline Form II of Compound 1 of any one of claims 1-18, wherein the crystalline Form II of Compound 1 is characterized by a TGA thermogram comprising a weight loss of about (0.360±0.018)% up to 220° C.

20. The crystalline Form II of Compound 1 of any one of claims 1-19, wherein the crystalline Form II of Compound 1 is obtained by phase equilibration in methyl iso-butyl acetone, n-butyl alcohol, methyl tert-butyl ether, ethyl acetate, iso-butyl acetate, iso-propyl acetate, or THF at about 25±5° C. or about 50±5° C.

21. The crystalline Form II of Compound 1 of any one of claims 1-20, wherein the crystalline Form II of Compound 1 is obtained by phase equilibration in dichloromethane and methyl ethyl ketone at about 50° C.

22. The crystalline Form II of Compound 1 of any one of claims 1-21, wherein the crystalline Form II of Compound 1 is substantially free of impurities.

23. The crystalline Form II of Compound 1 of any one of claims 1-22, wherein the crystalline Form II of Compound 1 is substantially free of amorphous forms of Compound 1.

24. A composition comprising the crystalline Form II of Compound 1 of claim 22 or 23, wherein the composition comprises greater than or equal to 90% by weight of the crystalline Form II of Compound 1.

25. The composition of claim 24, wherein the composition comprises greater than or equal to 95% by weight of the crystalline Form II of Compound 1.

26. The composition of claim 24 or 25, wherein the composition comprises greater than or equal to 99% by weight of the crystalline Form II of Compound 1.

27. A composition comprising the crystalline Form II of Compound 1 of claim 22 or 23, wherein the molar ratio of the amount of Form II of Compound 1 to the sum of amounts of other forms in the composition is equal to or greater than 90:10.

28. The composition of claim 27, wherein the molar ratio of the amount of Form II of Compound 1 to the sum of amounts of the other forms in the composition is equal to or greater than 95:5.

29. The composition of claim 27 or 28, wherein the molar ratio of the amount of Form II of Compound 1 to the sum of amounts of the other forms in the composition is equal to or greater than 99:1.

30. A crystalline Form III of Compound 1:

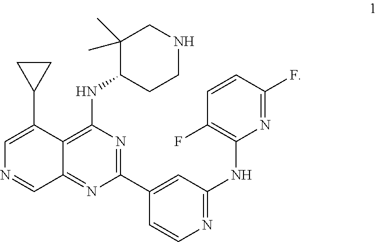 [SMILES: CC1(C)CNCC[C@@H]1Nc1nc(-c2ccnc(Nc3nc(F)ccc3F)c2)nc2cncc(C3CC3)c12]

31. The crystalline Form III of Compound 1 of claim 30, wherein the crystalline Form III of Compound 1 is characterized by an X-ray powder diffraction (XRPD) pattern substantially similar to that depicted in FIG. 4 when measured using CuK_{α} radiation.

32. The crystalline Form III of Compound 1 of claim 30 or 31, wherein the crystalline Form III of Compound 1 is characterized by an XRPD pattern comprising three or more characteristic peaks, expressed in degrees 2-theta (±0.2), of 4.3, 5.6, and 11.8 when measured using CuK_{α} radiation.

33. The crystalline Form III of Compound 1 of any one of claims 30-32, wherein the crystalline Form III of Compound 1 is characterized by an XRPD pattern comprising an additional characteristic peak, expressed in degrees 2-theta (±0.2), of 6.7 when measured using CuK_{α} radiation.

34. The crystalline Form III of Compound 1 of any one of claims 30-33, wherein the crystalline Form III of Compound 1 is characterized by a differential scanning calorimetry (DSC) thermogram substantially similar to that depicted in FIG. 5.

35. The crystalline Form III of Compound 1 of any one of claims 30-34, wherein the crystalline Form III of Compound 1 is characterized by a DSC thermogram comprising an endotherm comprising an onset temperature (Tₘ) of about 62.93±2.0° C.

36. The crystalline Form III of Compound 1 of any one of claims 30-35, wherein the crystalline Form III of Compound 1 is characterized by a DSC thermogram comprising a peak temperature (Tₘₐₓ) of about 93.28±2.0° C.

37. The crystalline Form III of Compound 1 of any one of claims 30-36, wherein the crystalline Form III of Compound 1 is characterized by a DSC thermogram comprising an enthalpy of the endothermic transition (AH) of about 80.80±4.0 J/g.

38. The crystalline Form III of Compound 1 of any one of claims 30-37, wherein the crystalline Form III of Compound 1 is characterized by a thermogravimetric analysis (TGA) thermogram substantially similar to that depicted in FIG. 6.

39. The crystalline Form III of Compound 1 of any one of claims 30-38, wherein the crystalline Form III of Compound 1 is characterized by a TGA thermogram comprising a weight loss of about (4.255±0.2)% up to 75° C.

40. The crystalline Form III of Compound 1 of any one of claims 30-39, wherein the crystalline Form III of Compound 1 is characterized by a TGA thermogram comprising a weight loss of about (1.240±0.060)% from 75° C. up to 175° C.

41. The crystalline Form III of Compound 1 of any one of claims 30-40, wherein the crystalline Form III of Compound 1 is obtained by phase equilibration in 1,4-dioxane at about 50±5° C.

42. The crystalline Form III of Compound 1 of any one of claims 30-41, wherein the crystalline Form III of Compound 1 is substantially free of impurities.

43. The crystalline Form III of Compound 1 of any one of claims 30-42, wherein the crystalline Form III of Compound 1 is substantially free of amorphous forms of Compound 1.

44. A composition comprising the crystalline Form III of Compound 1 of claim 42 or 43, wherein the composition comprises greater than or equal to 90% by weight of the crystalline Form III of Compound 1.

45. The composition of claim 44, wherein the composition comprises greater than or equal to 95% by weight of the crystalline Form III of Compound 1.

46. The composition of claim 44 or 45, wherein the composition comprises greater than or equal to 99% by weight of the crystalline Form III of Compound 1.

47. A composition comprising the crystalline Form III of Compound 1 of claim 42 or 43, wherein the molar ratio of the amount of Form III of Compound 1 to the sum of amounts of other forms in the composition is equal to or greater than 90:10.

48. The composition of claim 47, wherein the molar ratio of the amount of Form III of Compound 1 to the sum of amounts of the other forms in the composition is equal to or greater than 95:5.

49. The composition of claim 47 or 48, wherein the molar ratio of the amount of Form III of Compound 1 to the sum of amounts of the other forms in the composition is equal to or greater than 99:1.

50. A pharmaceutical composition comprising a crystalline form of any one of claims 1-23 or 30-43, or a composition of any one of claims 24-29 or 44-49, and a pharmaceutically acceptable excipient.

51. The pharmaceutical composition of claim 50, wherein the pharmaceutical composition is in the form of a topical formulation.

52. A method of preparing a crystalline Form II of Compound 1 of any one of claims 1-23, or a composition of any one of claims 24-29, the method comprising the steps of:
(1) providing a solution of Compound 1 in one or more solvents;
(2) precipitating the crystalline Form II of Compound 1 from the solution of Compound 1; and
(3) isolating the crystalline Form II of Compound 1.

53. The method of claim 52, wherein the solution of Compound 1 is provided by heating a mixture of solid Compound 1 and one or more solvents.

54. The method of claim 52 or 53, wherein at least one of the one or more solvents is a non-polar solvent.

55. The method of claim 52 or 53, wherein at least one of the one or more solvents is a polar solvent.

56. The method of any one of claims 52-55, wherein at least one of the one or more solvents is an aprotic solvent.

57. The method of any one of claims 52-55, wherein at least one of the one or more solvents is a protic solvent.

58. The method of claim 52 or 53, wherein at least one of the one or more solvents is acetonitrile, dichloromethane, dimethylformamide, 2-methoxy-ethanol, methyl iso-butyl ketone, acetone, n-butyl alcohol, methyl tert-butyl ether, ethyl acetate, heptane, isobutyl acetate, isopropyl acetate, methyl ethyl ketone, tetrahydrofuran, or dimethyl sulfoxide.

59. The method of any one of claims 52-58, wherein the solution of Compound 1 is saturated or supersaturated.

60. The method of any one of claims 52-59, wherein the precipitating step comprises one or more of phase equilibration, evaporation, anti-solvent addition, reverse addition, and cooling of a saturated solution.

61. The method of any one of claims 52-60, wherein the precipitating step comprises addition of one or more seed crystals of crystalline Form II of Compound 1.

62. The method of claim 60, wherein the precipitating step comprises phase equilibration in methyl iso-butyl acetone, n-butyl alcohol, methyl tert-butyl ether, ethyl acetate, iso-butyl acetate, iso-propyl acetate, or THF.

63. The method of claim 62, wherein the phase equilibration is performed between about 25±5° C. and about 50±5° C.

64. The method of claim 60, wherein the precipitating step comprises phase equilibration in dichloromethane or methyl ethyl ketone.

65. The method of claim 64, wherein the phase equilibration is performed at about 50±5° C.

66. The method of claim 60, wherein the precipitating step comprises anti-solvent addition to a solution of Compound 1 in dimethylformamide, 2-methoxy-ethanol, or dimethyl sulfoxide.

67. The method of claim 66, wherein the anti-solvent is methyl tert-butyl ether or acetonitrile.

68. The method of claim 60, wherein the precipitating step comprises reverse addition of an anti-solvent to a solution of Compound 1 in dimethylformamide, 2-methoxy-ethanol, or dimethyl sulfoxide.

69. The method of claim 68, wherein the anti-solvent is methyl tert-butyl ether or acetonitrile.

70. A method of preparing a crystalline Form III of Compound 1 of any one of claims 30-43, or a composition of any one of claims 44-49, the method comprising the steps of:
(1) providing a solution of Compound 1 in one or more solvents;
(2) precipitating the crystalline Form III of Compound 1 from the solution of Compound 1; and
(3) isolating the crystalline Form III of Compound 1.

71. The method of claim 70, wherein the solution of Compound 1 is provided by heating a mixture of solid Compound 1 and one or more solvents.

72. The method of claim 70 or 71, wherein at least one of the one or more solvents is a non-polar solvent.

73. The method of claim 70 or 71, wherein at least one of the one or more solvents is a polar solvent.

74. The method of any one of claims 70-73, wherein at least one of the one or more solvents is an aprotic solvent.

75. The method of any one of claims 70-73, wherein at least one of the one or more solvents is a protic solvent.

76. The method of claim 70 or 71, wherein at least one of the one or more solvents is 1,4-dioxane or n-propanol.

77. The method of any one of claims 70-76, wherein the solution of Compound 1 is saturated or supersaturated.

78. The method of claim 70, wherein the precipitating step comprises one or more of phase equilibration, evaporation, anti-solvent addition, reverse addition, and cooling of a saturated solution.

79. The method of any one of claims 70-78, wherein the precipitating step comprises addition of one or more seed crystals of crystalline Form III of Compound 1.

80. The method of claim 78, wherein the precipitating step comprises phase equilibration in 1,4-dioxane.

81. The method of claim 80, wherein the phase equilibration is performed at about 50±5° C.

82. A method for treating cancer in a subject in need thereof, comprising administering to the subject a crystalline form of any one of claims 1-23 or 30-43, a composition of any one of claims 24-29 or 44-49, or a pharmaceutical composition of claim 50 or 51.

83. The method of claim 82, wherein the cancer is basal cell carcinoma, a solid tumor, or acute myeloid leukemia.

84. A crystalline form of any one of claims 1-23 or 30-43, a composition of any one of claims 24-29 or 44-49, or a pharmaceutical composition of claim 50 or 51 for use in treating cancer in a subject in need thereof.

85. The crystalline form of claim 84, wherein the cancer is basal cell carcinoma, a solid tumor, or acute myeloid leukemia.

86. A use of a crystalline form of any one of claims 1-23 or 30-43, a composition of any one of claims 24-29 or 44-49, or a pharmaceutical composition of claim 50 or 51 for the treatment of cancer in a subject in need thereof.

87. Use of a crystalline form of any one of claims 1-23 or 30-43, a composition of any one of claims 24-29 or 44-49, or a pharmaceutical composition of claim 50 or 51 in the manufacture of a medicament for the treatment of basal cell carcinoma, solid tumors, or acute myeloid leukemia in a subject in need thereof.

88. The use of claim 86 or 87, wherein the cancer is basal cell carcinoma, a solid tumor, or acute myeloid leukemia.

89. A kit comprising a crystalline form of any one of claims 1-23 or 30-43, a composition of any one of claims 24-29 or 44-49, or a pharmaceutical composition of claim 50 or 51, and instructions for using the crystalline form, composition, or pharmaceutical composition.
